(19) United States
(12) Patent Application Publication
(10) Pub. No.: US 2026/0115335 A1
(43) Pub. Date: Apr. 30, 2026

(54) MOLECULES AND THEIR DERIVATIVES DIRECTED AGANIST CD45

(71) Applicant: Actinium Pharmaceuticals, Inc., New York, NY (US)

(72) Inventor: Dale L. LUDWIG, Rockaway, NJ (US)

(21) Appl. No.: 18/908,746

(22) Filed: Oct. 7, 2024

Related U.S. Application Data
(62) Division of application No. 16/840,806, filed on Apr. 6, 2020, now abandoned.
Application No. 16/840,806 is a continuation of application No. PCT/US2019/069041, filed on Dec. 31, 2019.
(60) Provisional application No. 62/799,990, filed on Feb. 1, 2019.

(51) Int. Cl. A61K 51/10; A61K 35/12; A61K 45/06; C07K 14/705; C07K 16/28

(52) U.S. Cl. CPC A61K 51/1027; A61K 35/12; C07K 14/70589; A61K 45/06; C07K 16/289

(57) ABSTRACT

The present disclosure provides epitopes of CD45, and binding agents such as antibodies, antibody fragments, peptides, and small molecules capable of binding to those epitopes. The present disclosure also provides methods of treating cancers, hematological diseases and disorders, and immune diseases and disorders, using these binding agents.

### CROSS-REFERENCE TO RELATED APPLICATION

This application is a continuation application of PCT/US2019/069041 filed Dec. 21, 2019, which claims the benefit of priority to U.S. Provisional Patent Application Ser. No. 62/799,990 filed Feb. 1, 2019, both of which are incorporated herein in their entirety.

### SEQUENCE LISTING

A Sequence Listing is submitted herewith in XML format under the file name “PT18048PCTCONDIV.xml,” created on Jan. 8, 2025, and having a size of 23,300 Bytes. The Sequence Listing is hereby incorporated by reference in its entirety.

### TECHNICAL FIELD

The present invention relates generally to epitopes of CD45, including epitopes of human CD45, and binding agents such as antibodies, antibody fragments, peptides, and small molecules capable of binding to CD45 and fragments thereof.

### BACKGROUND

CD45 is a type I transmembrane glycoprotein that is a member of the protein tyrosine phosphatase (PTP) family and plays a key role in T-cell and B-cell receptor signal transduction. CD45 controls activation of the Src family protein-tyrosine kinases Lck and Fyn. CD45 deficiency results in T- and B-lymphocyte dysfunction in the form of severe combined immune deficiency. It is also reported to play a significant role in autoimmune diseases and cancer as well as in infectious diseases including fungal infections (Penninger et al., 2001, CD45: new jobs for an old acquaintance, Nat. Immunol., 2(5):389-396). The primary ligands described for CD45 include galectin-1, CD1, CD2, CD3, CD4, TCR, CD22 and Thy-1.

Also known as leukocyte common antigen (LCA), T200, or Ly-5, CD45 consists of two intracellular phosphatase domains, a transmembrane domain and an extracellular domain. While both intracellular phosphatase domains are required for appropriate phosphate activity, only one has intrinsic kinase activity (Desai et al., 1994, The catalytic activity of the CD45 membrane-proximal phosphatase domain is required for TCR signaling and regulation, EMBO J. 13:4002-4010).

In general, all cells of hematopoietic origin, with the exception of mature erythrocytes and platelets, express at least one isoform of CD45. High expression of CD45 is seen with most acute lymphoid and myeloid leukemias. Since CD45 is not found on tissues of non-hematopoietic origin, its specific expression in leukemia has made it a good target for developing therapeutics, including immunotherapeutics and radio-immunotherapeutics. For example, CD45 is expressed at a density of approximately 200,000 to 300,000 sites per cell on circulating leukocytes and malignant B cells. One particular anti-CD45 antibody, BC8, has been explored as a candidate immunotherapeutic agent alone and in combination with chemotherapy or total body irradiation in the treatment of leukemias. The use of BC8 labelled with ¹³¹I- for the treatment of subjects needing bone marrow transplant has also been explored (see International Publication No. WO 2017/155937).

CD45 exists as multiple isoforms due to alternative splicing of three of the 34 exons (exons 4, 5, and 6, designated A, B, and C; see FIG. 1) in the extracellular domain (Streuli et al., 1987 Differential usage of three exons generates at least five different mRNAs encoding human leukocyte common antigens, J. Exp. Med. 166:1548-1566; Chang et al., 2016, Initiation of T cell signaling by CD45 segregation at ‘close-contacts’, Nat. Immunol. 17(5):574-582). These three exons encode multiple sites of O-linked glycosylation and are variably modified by sialic acid. As a result, the various isoforms differ substantially in size (391 to 552 amino acids; molecular weight ranging from 180)-240 kDa), shape, and negative charge. The remaining membrane proximal extracellular domain is heavily N-glycosylated and contains a cysteine-rich spacer region followed by three fibronectin type III repeats (FIG. 2).

While eight isoforms of CD45 are possible, only six are identified in humans: RO (absent all three exons), RA (exon A), RB (exon B), RAB (exons A and B), RBC (exons B and C), and RABC (exons A, B, and C). These different isoforms are differentially expressed on subpopulations of B- and T-cell lymphocytes and are specific to the activation and maturation state of the cell. For example, CD45-RA and CD45-RB are expressed on naïve T-cells, while CD45-RO is expressed on activated T-cells, some B-cell subsets, activated monocytes/macrophages, and granulocytes, and CD45-RABC is preferentially expressed on B-cells (Hermiston et al., 2003, CD45: A critical regulator of signaling thresholds in immune cells, Ann. Rev. Immunol., 21:107-137).

Antibodies that selectively recognize various isoforms of CD45 have been identified. In addition, monoclonal antibodies (mAbs) that bind an epitope common to all the different isoforms have also been identified. For example, the anti-CD45 murine antibody BC8 recognizes all the human isoforms of the CD45 antigen. Identification of the binding site for such a pan anti-CD45 antibody, as well as additional epitopes of CD45 and binding agents such as antibodies, antibody fragments, peptides, and small molecules capable of binding to CD45 are the subject of the present disclosure. The potential applications of these binding agents for clinical and therapeutic use are also a subject of the present disclosure.

### SUMMARY

The present invention relates to regions (epitopes) of CD45, binding agents that recognize the disclosed epitopes, therapeutic methods of using these binding agents, and compositions comprising these binding agents. Binding agents may include antibodies, antibody fragments, peptides, and small molecules.

The invention relates to epitopes of CD45 within the region identified as the cysteine-rich spacer region, and binding agents such as antibodies, antibody fragments, peptides, and small molecules that bind within that region.

The invention also relates to epitopes of CD45 within the region conserved among all isoforms of CD45, such as a region near the N-terminal of the cysteine-rich spacer region, and binding agents that bind within that region.

The invention further relates to epitopes of CD45 that are within a specific 33 amino acid segment of the cysteine-rich spacer region of CD45, wherein the cysteine-rich spacer region is defined by residues between and including C228 and C288 in humans, and binding agents that bind to or within that region.

The invention further relates to a conformational epitope in the cysteine-rich spacer region of CD45 comprising at least two, three, four, or five amino acid residues selected from V254, N257, E259, N267, N268, H285, and N286, and binding agents that bind to those amino acids, and/or to the conformational region of CD45 comprising at least two, three, four, or five of those amino acids.

The invention further relates to a conformational epitope in the cysteine-rich spacer region of CD45 comprising the six amino acid residues V254, N257, E259, N267, H285, and N286, and binding agents that bind to those amino acids, and/or to the conformational region of CD45 comprising those amino acids.

The invention further relates to a conformational epitope in the cysteine-rich spacer region of CD45 comprising the seven amino acid residues V254, N257, E259, N267, N268, H285, and N286, and binding agents that bind to those amino acids, and/or to the conformational region of CD45 comprising those amino acids.

The invention also relates to binding agents that bind to any of the epitopes disclosed herein. The binding agents can be characterized by their ability to cross-block the binding of at least one antibody disclosed herein to CD45 and/or to be cross-blocked from binding CD45 by at least one antibody disclosed herein. An exemplary antibody includes the monoclonal antibody BC8.

The binding agents may include antibodies, antibody fragments, peptides, and small molecules that bind to CD45 or a region or fragment thereof. As such, the invention also relates to antibodies such as BC8, and fragments of an antibody or peptides that may bind to any of the epitopes of CD45 disclosed herein. Exemplary antibody fragments and peptides may comprise sequences and structures defined by, or comprising any of SEQ ID NOS: 2-15 and 17, or encoded by any portion of the polynucleotide sequences of SEQ ID NOS: 16 and 18.

The invention further relates to polynucleotides encoding a monoclonal antibody, antibody fragment, or peptide that binds to amino acids in the cysteine-rich spacer region of CD45. The amino acids may comprise at least two, three, four, or five amino acids in the d1 region of human CD45 (SEQ ID NO: 19); at least two, three, four, or five amino acids in the region 228-288 of human CD45 (SEQ ID NO: 20); at least two, three, four, or five amino acids in the region 254-286 of human CD45 (SEQ ID NO: 21); at least two, three, four, or five of the amino acids V254, N257, E259, N267, N268, H285, N286 of human CD45 (see SEQ ID NO: 1); amino acids V254, N257, E259, N267, H285, N286 of human CD45 (see SEQ ID NO: 1); and amino acids V254, N257, E259, N267, N268, H285, N286 of human CD45 (see SEQ ID NO: 1).

The invention further relates to polypeptide constructs comprising two, three, or four regions of polypeptide structure (i.e., portions of secondary structure) representing the cysteine-rich spacer region of CD45, or a portion thereof, and binding agents that specifically bind thereto. The constructs may comprise one or more polypeptide fragments and may further comprise one or more disulfide bonds. Exemplary polypeptide constructs may comprise sequences and structures defined by any of SEQ ID NOS: 19-21.

The CD45 may be from Chordata (i.e., members of Classes Osteichythyes (bony fish), Amphibia (amphibians), Reptilia (reptiles), Aves (birds), and Mammalia (mammals)), and all Orders and Families encompassed therein. The CD45 may be mammalian, such as from any of humans, cattle, swine, horses, sheep, goats, dogs and cats. According to certain aspects, the CD45 may be human CD45.

The invention also relates to methods of obtaining epitopes suitable for use as immunogens for generating, in mammals, binding agents, such as antibodies, antibody fragments, peptides, and small molecules capable of binding specifically to CD45. According to certain aspects, the binding agents disclosed herein are capable of inhibiting CD45 activity in vivo.

The invention further relates to compositions comprising any of these binding agents and methods useful in the treatment of a malignant or non-malignant hematological disease or disorder, a proliferative disorder, such as a cancer or solid tumor, and/or to effect lymphodepletion or myeloablation.

The objects of the present invention will be realized and attained by means of the combinations specifically outlined in the appended claims. The foregoing general description and the following detailed description and examples of this invention are provided to illustrate various aspects of the present invention, and by no means are to be viewed as limiting any of the described embodiments.

### BRIEF DESCRIPTION OF THE DRAWINGS

FIG. 1 shows a schematic diagram of exon usage in various isoforms of CD45 produced by differential splicing of the human CD45 gene.

FIG. 2 shows a schematic diagram of several CD45 isoforms.

FIG. 3 provides the amino acid sequence of the full-length CD45 protein (CD45-RABC isoform; SEQ ID NO. 1), wherein the Fibronectin type-III domains are highlight in the extracellular region (underlined), and the Tyrosine-protein phosphatase 1 and Tyrosine-protein phosphatase 2 domains are highlighted in the intracellular region (single and double underlining, respectively).

FIG. 4A provides a schematic diagram of the full-length human CD45 sequence: SP, secretion signal peptide; TM, transmembrane domain; P1 and P2, tyrosine-protein phosphatase catalytic domains; exons A, B and C, which are spliced out in the RO isoform; domain d1, the cysteine-rich spacer region, and domains d2-d4, the Fibronectin type-III domains.

FIG. 4B provides a cartoon representation of a crystal structure of the d1-d4 region of CD45 (pdb-5FMV) with individual domains shaded as in FIG. 4A.

FIG. 5 provides the sequence of the complementarity determining regions (CDRs), framework regions, and variable domain sequences of the light chain (VL; SEQ ID NO. 2) and the heavy chain (VH; SEQ ID NO. 3) of the anti-CD45 mAb BC8, wherein the CDRs are in bold and underlined.

FIG. 6A provides amino acid sequences comprising the CDRs and an N-terminal portion of the light chain and the heavy chain of the anti-CD45 mAb BC8 (SEQ ID NOS. 4-11).

FIG. 6B provides amino acid sequences for mouse and human IgG1 CH1 (SEQ ID NOS. 12 and 13, respectively), and human kappa (SEQ ID NO: 14).

FIG. 7 provides the amino acid (SEQ ID NO: 15) and nucleotide (SEQ ID NO: 16) sequence of the light chain of the anti-CD45-immunoglobulin BC8.

FIG. 8 provides the amino acid (SEQ ID NO: 17) and nucleotide (SEQ ID NO: 18) sequence of the heavy chain of the anti-CD45-immunoglobulin BC8.

FIGS. 9A-9D show representative data for optimization of the protein concentration of various monoclonal anti-CD45 antibodies for use in flow cytometry experiments, wherein each antibody was tested for immunoreactivity against cells expressing mutant CD45 proteins (alanine scanning library) or vector. FIG. 9A shows optimization for the BC8 monoclonal antibody, FIG. 9B shows optimization for the ab8216 monoclonal antibody, FIG. 9C shows optimization for the 2D1 monoclonal antibody, and FIG. 9D shows optimization for the BC8 antigen binding fragment.

FIG. 10 shows binding of monoclonal antibodies (Mab) against CD45 to each mutant clone (mutant CD45) in the alanine scanning library as determined by high-throughput flow cytometry, wherein the mean binding value was plotted as a function of expression (represented by control reactivity). A threshold (dashed lines) of >50% WT binding to control MAb and <15% WT binding to test MAbs was applied to identify critical residues.

FIG. 11A provides the amino acid sequence (SEQ ID NO: 19) for the cysteine-rich spacer region of CD45 (d1).

FIG. 11B provides the amino acid sequence (SEQ ID NO: 20) for the cysteine-rich spacer region of CD45 defined by cysteine residues 228 and 288.

FIG. 11C provides the amino acid sequence (SEQ ID NO: 21) for a 33 amino acid stretch in the cysteine-rich spacer region of CD45.

FIG. 11D shows the sequence homology between human and monkey sequence in the cysteine-rich spacer region of CD45.

FIG. 12A shows the cartoon representation of the d1-d4 region of CD45 based on the crystal structure (PDB-5FMV) with the critical residues of the CD45 epitope mapped thereon as spheres.

FIG. 12B shows a close-up of the d1 region (A) of the 3D structure shown in FIG. 11A with specific amino acids in the conformational epitope labelled.

FIG. 13 shows representative flow cytometry results for BC8 antibody immunophenotyping with B Lymphocytes demonstrating the pan-specificity of the antibody.

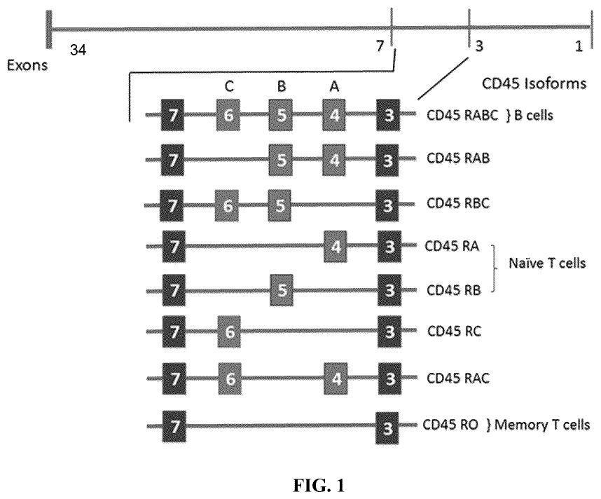

### BRIEF DESCRIPTION OF THE SEQUENCES

SEQ ID NO:1 is the amino acid sequence of the RABC isoform of the human CD45 protein.

SEQ ID NO:2 is the amino acid sequence of the variable domain of the light chain of the anti-CD45 murine immunoglobulin BC8.

SEQ ID NO:3 is the amino acid sequence of the variable domain of the heavy chain of the anti-CD45 murine immunoglobulin BC8.

SEQ ID NO:4 is the amino acid sequence of CDR1 of the light chain of the anti-CD45 murine immunoglobulin BC8.

SEQ ID NO:5 is the amino acid sequence of CDR2 of the light chain of the anti-CD45 murine immunoglobulin BC8.

SEQ ID NO:6 is the amino acid sequence of CDR3 of the light chain of the anti-CD45 murine immunoglobulin BC8.

SEQ ID NO:7 is the amino acid sequence of CDR1 of the heavy chain of the anti-CD45 murine immunoglobulin BC8.

SEQ ID NO:8 is the amino acid sequence of CDR2 of the heavy chain of the anti-CD45 murine immunoglobulin BC8.

SEQ ID NO:9 is the amino acid sequence of CDR3 of the heavy chain of the anti-CD45 murine immunoglobulin BC8.

SEQ ID NO:10 is the amino acid sequence of a portion of the anti-CD45 murine immunoglobulin BC8 comprising the N-terminus of the light chain.

SEQ ID NO:11 is the amino acid sequence of a portion of the anti-CD45 murine immunoglobulin BC8 comprising the N-terminus of the heavy chain.

SEQ ID NO:12 is the amino acid sequence of the IgG1 CH1 domain of the heavy chain of the anti-CD45 murine immunoglobulin BC8.

SEQ ID NO:13 is the amino acid sequence of the human kappa region, i.e., light chain constant region of a human immunoglobulin.

SEQ ID NO:14 is the amino acid sequence of the IgG1 CH1 domain of the heavy chain of a human immunoglobulin.

SEQ ID NO:15 is the amino acid sequence of the light chain of the anti-CD45 murine immunoglobulin BC8.

SEQ ID NO:16 is the nucleotide sequence of the light chain of the anti-CD45 murine immunoglobulin BC8.

SEQ ID NO:17 is the amino acid sequence of the heavy chain of the anti-CD45 murine immunoglobulin BC8.

SEQ ID NO:18 is the nucleotide sequence of the heavy chain of the anti-CD45 murine immunoglobulin BC8.

SEQ ID NO:19 is the amino acid sequence of the cysteine-rich spacer region of CD45 (d1).

SEQ ID NO:20 is the amino acid sequence for an epitope of CD45 in the cysteine-rich spacer region defined by cysteine residues 228 and 288.

SEQ ID NO:21 is the amino acid sequence for an epitope of CD45 in the cysteine-rich spacer region.

### DETAILED DESCRIPTION

The present disclosure provides epitopes of CD45, and binding agents such as antibodies, antibody fragments, peptides, and small molecules capable of binding to those epitopes. Moreover, the present disclosure provides methods of treating cancers, hematological diseases and disorders, and immune diseases and disorders, using these epitopes and binding agents.

### Definitions

In this application, certain terms are used which shall have the meanings set forth as follows.

The singular forms “a,” “an,” “the” and the like include plural referents unless the context clearly dictates otherwise. Thus, for example, reference to “an” antibody includes both a single antibody and a plurality of different antibodies.

The term “about” when used before a numerical designation, e.g., temperature, time, amount, and concentration, including a range, indicates approximations which may vary by +10%, +5%, or +1%.

“Comprising” or “comprises” is intended to mean that the compositions and methods include the recited elements, but do not exclude others. “Consisting essentially of” when used to define compositions and methods, shall mean excluding other elements of any essential significance to the combination for the stated purpose. Thus, a method consisting essentially of the elements as defined herein would not exclude other steps or composition that do not materially affect the basic and novel characteristic(s) of the claimed invention.

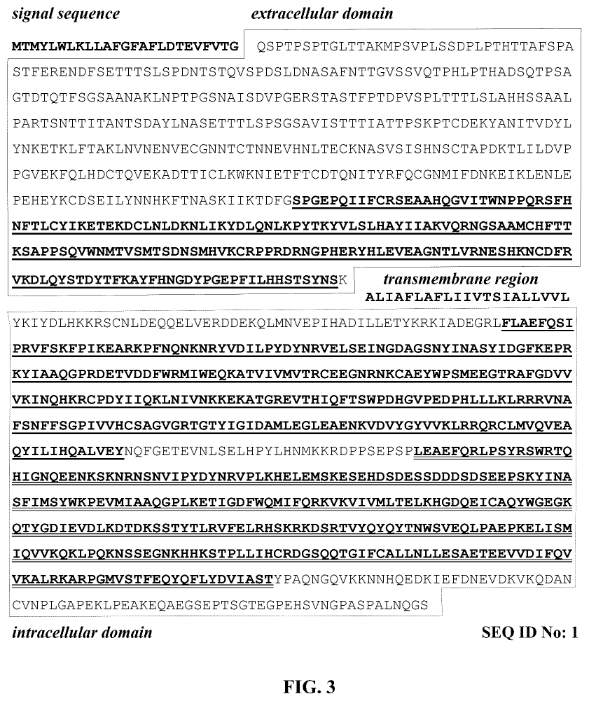

“CD45” refers to the human CD45 protein (synonyms: Protein tyrosine phosphatase, receptor type, C; also known as PTPRC). Human CD45 has the amino acid sequence shown in SEQ ID NO: 1 (FIG. 3).

As used herein, the term “antibody” includes, without limitation, (a) an immunoglobulin molecule comprising two heavy chains and two light chains and which recognizes an antigen; (b) polyclonal and monoclonal immunoglobulin molecules (e.g., Mab); (c) monovalent and divalent fragments thereof (e.g., di-Fab), and (d) bi-specific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. Antibodies can be both naturally occurring and non-naturally occurring (e.g., IgG-Fc-silent). Furthermore, antibodies include chimeric antibodies, wholly synthetic antibodies, single chain antibodies, and fragments thereof. Antibodies may be human, humanized or nonhuman.

“Monoclonal antibody” refers to a preparation of antibody molecules of single molecular composition. A monoclonal antibody composition displays a single binding specificity and affinity for a particular epitope, or in a case of a bispecific monoclonal antibody, a dual binding specificity to two distinct epitopes. “Monoclonal antibody” therefore refers to an antibody population with single amino acid composition in each heavy and each light chain, except for possible well-known alterations such as removal of C-terminal lysine from the antibody heavy chain. Monoclonal antibodies may have heterogeneous glycosylation within the antibody population. Monoclonal antibodies may be monospecific or multispecific, or monovalent, bivalent or multivalent. A bispecific antibody is included in the term monoclonal antibody.

As used herein, an “anti-CD45 antibody” is an antibody that binds to any available epitope of CD45. According to certain aspects, the anti-CD45 antibody binds to the epitope recognized by the monoclonal antibody “BC8.” BC8 is known, as are methods of making it. According to certain other aspects, the anti-CD antibody binds to the epitope(s) of CD45 disclosed herein.

An “epitope” generally refers to the target molecule site that is capable of being recognized by, and bound by, an antibody. For a protein epitope, for example, this may refer to the amino acids (and particularly their side chains) that are bound by the antibody. Protein epitopes can be divided into two classes, “linear epitopes” which comprise continuous stretches of amino acids and “conformational epitopes” which comprise discontinuous amino acids in a protein sequence that are brought into proximity with one another by the three-dimensional structure of the protein.

“Humanized antibody” refers to an antibody in which the antigen binding sites are derived from non-human species and the variable region frameworks are derived from human immunoglobulin sequences. Humanized antibodies may include substitutions in the framework regions so that the framework may not be an exact copy of expressed human immunoglobulin or germline gene sequences.

“Chimeric antibody” refers to an antibody having heavy and light chain variable regions in which both the framework and antigen binding sites are derived from sequences of one species, typically mouse, rat, or rabbit and the heavy and light chain constant regions are derived from another species, typically human.

“Human antibody” refers to an antibody having heavy and light chain variable regions in which both the framework and the antigen binding sites are derived from sequences of human origin. If the antibody contains a constant region, the constant region is also derived from sequences of human origin.

A human antibody comprises heavy or light chain variable regions having variable domain sequences that are “derived from” sequences of human origin wherein the variable regions of the antibody are obtained from a system that uses human germline immunoglobulin or rearranged immunoglobulin genes. Such systems include human immunoglobulin gene libraries displayed on phage, and transgenic non-human animals such as mice carrying human immunoglobulin loci. A human antibody may contain amino acid differences when compared to the human germline immunoglobulin or rearranged immunoglobulin genes due to for example naturally occurring somatic mutations or intentional introduction of substitutions in the framework or antigen binding site, or both. Typically, human antibody refers to an antibody having at least about 80%, 81%, 82%, 83%, 84%, 85%, 86%, 87%, 88%, 89%, 90%, 91%, 92%, 93%, 94%, 95%, 96%, 97%, 98%, 99% or 100% identical amino acid sequence to an amino acid sequence encoded by human germline immunoglobulin or rearranged immunoglobulin genes.

“Immunoreactivity” refers to a measure of the ability of an immunoglobulin to recognize and bind to a specific antigen.

As used herein, the term “binding agent” or “agent” may be taken to include any antibody, antibody fragment, peptide, or small molecule that may bind to any of the epitopes disclosed herein. Moreover, the binding agents can be characterized by their ability to cross-block the binding of at least one antibody disclosed herein to CD45 and/or to be cross-blocked from binding CD45 by at least one antibody disclosed herein.

“Pharmaceutically acceptable salt” refers to acid addition salts of basic compounds, e.g., those compounds including a basic amino group, and to basic salts of acidic compounds, e.g., those compounds including a carboxyl group, and to amphoteric salts of compounds that include both an acidic and a basic moiety, such that these salts are suitable for administration in vivo, preferably to humans. Various organic and inorganic acids may be used for forming acid addition salts. Pharmaceutically acceptable salts are derived from a variety of organic and inorganic counter ions well known in the art. Pharmaceutically acceptable salts include, when the molecule contains a basic functionality, by way of example only, hydrochloride, hydrobromide, tartrate, mesylate, acetate, maleate, oxalate and the like, and when the molecule contains an acidic functionality, by way of example only, sodium, potassium, calcium, magnesium, ammonium, tetraalkylammonium, N-methylmorpholinium, and the like.

As used herein, “cancer” includes, without limitation, a solid cancer (e.g., a tumor) and a hematologic malignancy.

A “hematologic malignancy”, also known as a blood cancer, is a cancer that originates in blood-forming tissue, such as the bone marrow or other cells of the immune system. Hematologic malignancies include, without limitation, leukemias (such as acute myeloid leukemia (AML), acute promyelocytic leukemia, acute lymphoblastic leukemia (ALL), acute mixed lineage leukemia, chronic myeloid leukemia, chronic lymphocytic leukemia (CLL), hairy cell leukemia and large granular lymphocytic leukemia), myelodysplastic syndrome (MDS), myeloproliferative disorders (polycythemia vera, essential thrombocytosis, primary myelofibrosis and chronic myeloid leukemia), lymphomas, multiple myeloma, MGUS and similar disorders, Hodgkin's lymphoma, non-Hodgkin lymphoma (NHL), primary mediastinal large B-cell lymphoma, diffuse large B-cell lymphoma, follicular lymphoma, transformed follicular lymphoma, splenic marginal zone lymphoma, lymphocytic lymphoma. T-cell lymphoma, and other B-cell malignancies.

As used herein, a subject's “peripheral blood lymphocytes” shall mean the mature lymphocytes circulating in the subject's blood. Examples of peripheral blood lymphocytes include, without limitation, peripheral blood T-cells, peripheral blood NK cells and peripheral blood B cells. A subject's peripheral blood lymphocyte population is readily measurable. Thus, by measuring a decrease in the level of at least one type of peripheral blood lymphocyte following a depleting event (e.g., the administration of a low ¹³¹I-BC8 dose), one can easily determine that lymphodepletion has occurred in a subject.

“Solid cancers” include, without limitation, bone cancer, pancreatic cancer, skin cancer, cancer of the head or neck, cutaneous or intraocular malignant melanoma, uterine cancer, ovarian cancer, prostate cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, pediatric tumors, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally-induced cancers including those induced by asbestos.

As used herein, the term “subject” includes, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 60 years or older, 65 or older, 70 or older, 75 or older, 80 or older, 85 or older, or 90 or older. Alternatively, the subject can be 50 years or younger, 45 or younger, 40 or younger, 35 or younger, 30 or younger, 25 or younger, or 20 or younger. For a human subject afflicted with cancer, the subject can be newly diagnosed, or relapsed and/or refractory, or in remission. “Patient” and “subject” are used interchangeably herein.

As used herein, a “radioisotope” can be an alpha-emitting isotope, a beta-emitting isotope, and/or a gamma-emitting isotope. Examples of radioisotopes include the following: ³²P, ²¹¹At, ¹³¹I, ¹³⁷Cs, ⁹⁰Y, ¹⁷⁷Lu, ¹⁸⁶Re, ¹⁸⁸Re, ⁸⁹Sr, ¹⁵³Sm, ²²⁵Ac, ²¹³Bi, ²¹³Po, ²¹²Bi, ²²³Ra, ²²⁷Th, ¹⁴⁹Th, ¹⁶⁴Cu, ²¹²Pb, ⁸⁹Zr, ⁶⁸Ga, and ¹⁰³Pd. Thus, the radiolabeled binding agents (Agent) envisioned in this invention include, without limitation, ³²P-Agent, ²¹¹At-Agent, ¹³¹I-Agent, ¹³⁷Cs-Agent, ⁹⁰Y-Agent, ⁸⁹Sr-Agent, ¹⁵³Sm-Agent, ³²P-Agent, ²²⁵Ac-Agent, ²¹³Bi-Agent, ²¹³Po-Agent, ²¹¹At-Agent, ²¹²Bi-Agent, ²¹³Bi-Agent, ²²³Ra-Agent, ²²⁷Th-Agent, ¹⁴⁹Tb-Agent, ¹³¹I-Agent, ¹³⁷Cs-Agent, ²¹²Pb-Agent, and ¹⁰³Pd-Agent, where “Agent” may be taken to represent an intact antibody that binds to CD45 and the specific epitopes of CD45 disclosed herein, such as BC8, and further to antibody fragments, peptides, and small molecules that bind to CD45 and the specific epitopes of CD45 disclosed herein.

Methods of labeling BC8 with ¹³¹I or ²²⁵Ac are known. These methods are described, for example, in International Publication Nos. WO 2017/155937 and WO 2019/027973, and may be further applicable to any of the binding agents disclosed herein.

As used herein, “treating” a subject afflicted with a cancer shall include, without limitation, (i) slowing, stopping or reversing the cancer's progression, (ii) slowing, stopping or reversing the progression of the cancer's symptoms, (iii) reducing the likelihood of the cancer's recurrence, and/or (iv) reducing the likelihood that the cancer's symptoms will recur. According to certain preferred aspects, treating a subject afflicted with a cancer means (i) reversing the cancer's progression, ideally to the point of eliminating the cancer, and/or (ii) reversing the progression of the cancer's symptoms, ideally to the point of eliminating the symptoms, and/or (iii) reducing or eliminating the likelihood of relapse (i.e., consolidation, which ideally results in the destruction of any remaining cancer cells).

“Therapeutically effective amount” refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body.

“Inhibits growth” refers to a measurable decrease or delay in the growth of a malignant cell or tissue (e.g., tumor) in vitro or in vivo when contacted with a therapeutic or a combination of therapeutics or drugs, when compared to the decrease or delay in the growth of the same cells or tissue in the absence of the therapeutic or the combination of therapeutic drugs. Inhibition of growth of a malignant cell or tissue in vitro or in vivo may be at least about 10%, 20%, 30%, 40%, 50%, 60%, 70%, 80%, 90%, 95%, 96%, 97%, 98%, 99%, or 100%.

Unless otherwise defined, all technical and scientific terms used herein have the same meaning as commonly understood by one of ordinary skill in the art to which the present invention belongs. Although methods and materials similar or equivalent to those described herein can be used in the practice or testing described herein, suitable methods and materials are described below.

### Detailed Description

Disclosed herein are regions (epitopes) of CD45, and agents capable of specific binding to those epitopes. Moreover, methods for identifying those regions within CD45 are disclosed, as well as methods for generating binding agents specific to those regions. Compositions comprising the identified binding agents, i.e., agents capable of specifically binding to the epitopes of CD45, are disclosed, and methods of using those compositions to treat malignant and non-malignant diseases and disorders, such as a malignant and non-malignant hematological diseases and disorders, and to affect lymphodepletion or myeloablation, are disclosed. These compositions may be used alone or in combination with other therapies and agents, such as chemotherapies, adoptive cell therapies, or additional immunotherapies.

The binding agents disclosed herein are specific for an epitope of CD45. The epitope may be an epitope common to all isoforms of CD45, such as all isoforms of human CD45. As such, the epitope may comprise a linear or conformational epitope recognized by a pan-anti-CD45 antibody. The binding agents may include intact antibodies, antibody fragments, peptides, and/or small molecules capable of binding any of the epitopes of CD45 disclosed herein.

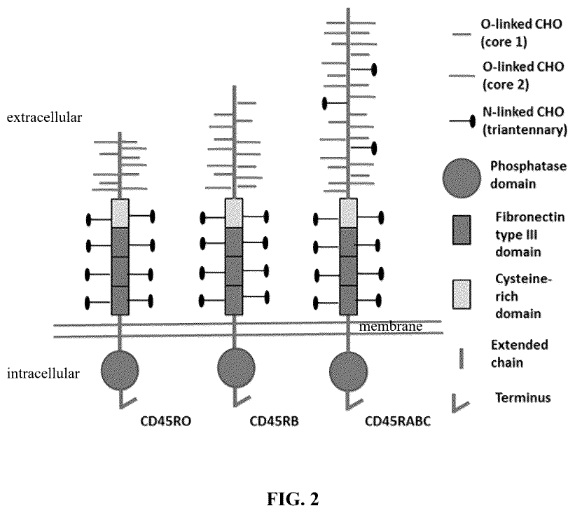

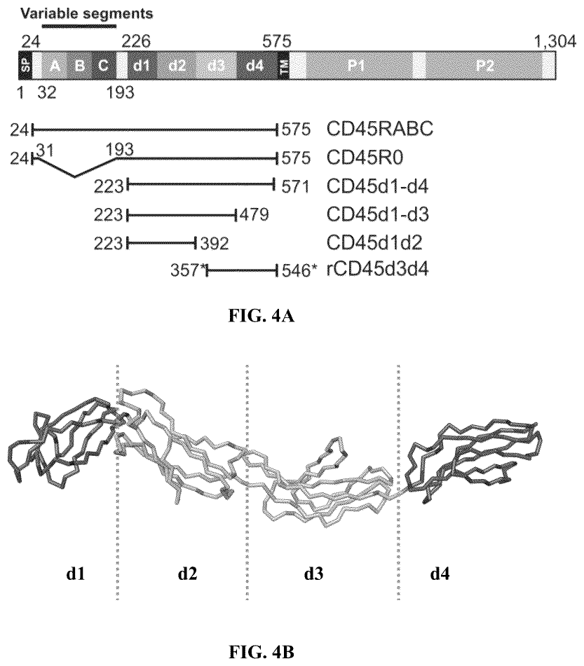

Among several clones of the anti-CD45 murine antibody, BC8 recognizes all human isoforms of the CD45 antigen, and weakly cross-reacts with monkey CD45. While the amino terminal portion of the extracellular domain of CD45 imparts isoform variability (see FIG. 2), the remaining portion of the receptor contains a short cysteine-rich spacer region, followed by a series of Fibronectin-type III domains. The predicted binding site for a pan-anti-CD45 antibody such as BC8 is likely in this cysteine-rich spacer region and/or in the fibronectin type III repeats of the extracellular region of CD45 (see FIG. 4A, 4B).

The presently disclosed invention defines an epitope comprising amino acids in the d1 region of the CD45 protein which encompasses the cysteine-rich spacer region. The invention further includes an epitope comprising all or a portion of the amino terminal (N-terminal) region of the cysteine-rich spacer region of CD45. This N-terminal region was found to be important for the generation of antibodies, antibody fragments, peptides, and small molecules that can bind to all isoforms of CD45, and specifically as important for binding of BC8. Further, the invention defines the cysteine-rich spacer region as important in the selection and screening of pan-CD45 molecules, recognizing the presence of three conserved fibronectin type-III (FnIII) domains across species and the broad expression of FnIII domains in many cell-surface associated receptors in mammals and other species.

The epitopes of CD45 disclosed herein were identified using an epitope mapping and protein engineering platform that introduced hundreds of specific mutations into the CD45 protein, i.e., alanine scanning. These mutant CD45 proteins were then tested individually for their effects in human cells by high throughput flow cytometry, i.e., testing the ability of an anti-CD45 antibody to bind to the cells expressing the mutant CD45 proteins. By combining large-scale mutagenesis with rapid cellular testing of natively folded proteins, critical domains were identified in the structurally complex CD45 protein. The binding site of a pan-anti-CD45 antibody such as BC8 was mapped to amino acids in the cysteine-rich spacer region of the CD45 protein.

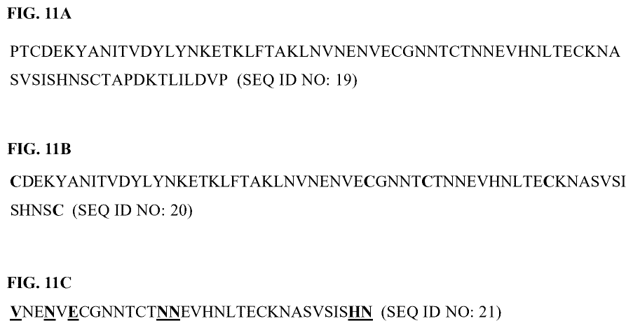

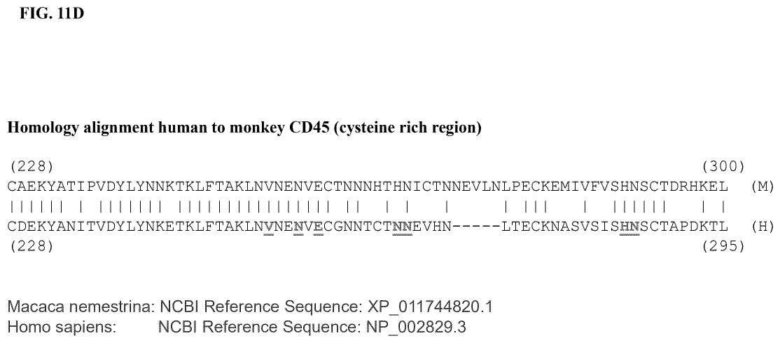

Accordingly, the present invention provides linear and conformational epitopes of CD45 that lie within or comprise a portion of the protein region defined by the sequences SEQ ID NOS: 19-21 (see FIG. 11A-11C). Shown in FIG. 11A is the sequence of the cysteine-rich spacer region (d1) of human CD45, including amino acids residues 226 to 300 of the human CD45 protein (SEQ ID NO: 19). Shown in FIG. 11B is a portion of the cysteine-rich spacer region defined by cysteine residues 228 to 288 of the human CD45 protein (SEQ ID NO: 20). Shown in FIG. 11C is a 33 amino acid region from within the cysteine-rich spacer region of CD45 (SEQ ID NO: 21), wherein the region comprises the six (V254, N257, E259, N267, H285, and N286) or seven (V254, N257, E259, N267, N268, H285, and N286) amino acids that are part of a conformational epitope (see Table 1).

TABLE 1
Amino Acid residues of the Conformational Epitope

| Amino Acid # | Identity |
|---|---|
| 254 | Valine |
| 257 | Asparagine |
| 259 | Glutamic Acid |
| 267 | Asparagine |
| 268 | Asparagine |
| 285 | Histidine |
| 386 | Asparagine |

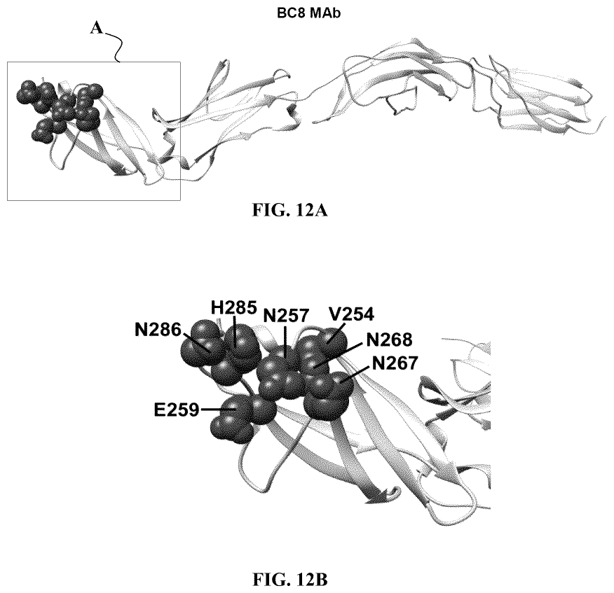

The amino acids in the cysteine-rich spacer region of CD45 found to be key residues for binding of the pan-anti-CD45 antibodies, as tested herein, are characterized by a clustered folding of B-sheets in the CD45 protein (see FIGS. 12A and 12B). This cluster of protein secondary structure defines a conformational epitope. While this region is common among CD45 isoforms, it is not part of the larger conserved Fibronectin domain region (d2-d4 of FIG. 4A, 4B). This region shows poor conservation between human and lower species, and only about 50% conservation across the domain between human and monkey (see FIG. 11D). The amino acids that are part of the conformation epitope disclosed herein, however, are well conserved between human and monkey, explaining the cross-reactivity between species of the pan-anti-CD45 antibody used to define the epitope of this present invention (i.e., BC8).

Accordingly, the present invention includes epitopes of CD45 comprising any of: at least two, three, four, or five amino acids in the d1 region of human CD45 (SEQ ID NO: 19); at least two, three, four, five, or six amino acids in the region 228-288 of human CD45 (SEQ ID NO: 20); at least two, three, four, five, or six amino acids in the region 254-286 of human CD45 (SEQ ID NO: 21); at least two, three, four, five, or six of the amino acids V254, N257, E259, N267, N268, H285, N286 of human CD45 (see SEQ ID NO: 1); the amino acids V254, N257, E259, N267, H285, N286 of human CD45 (see SEQ ID NO: 1); or the amino acids V254, N257, E259, N267, N268, H285, N286 of human CD45 (see SEQ ID NO: 1).

The epitopes may be conformational epitopes comprising a three-dimensional structure defined by a portion of the cysteine-rich spacer region of CD45, such as by any of the portions listed hereinabove.

The present invention is further directed to binding agents that may bind to any of the epitopes of CD45 disclosed herein. The binding agents may include intact antibodies, antibody fragments, peptides, and/or small molecules.

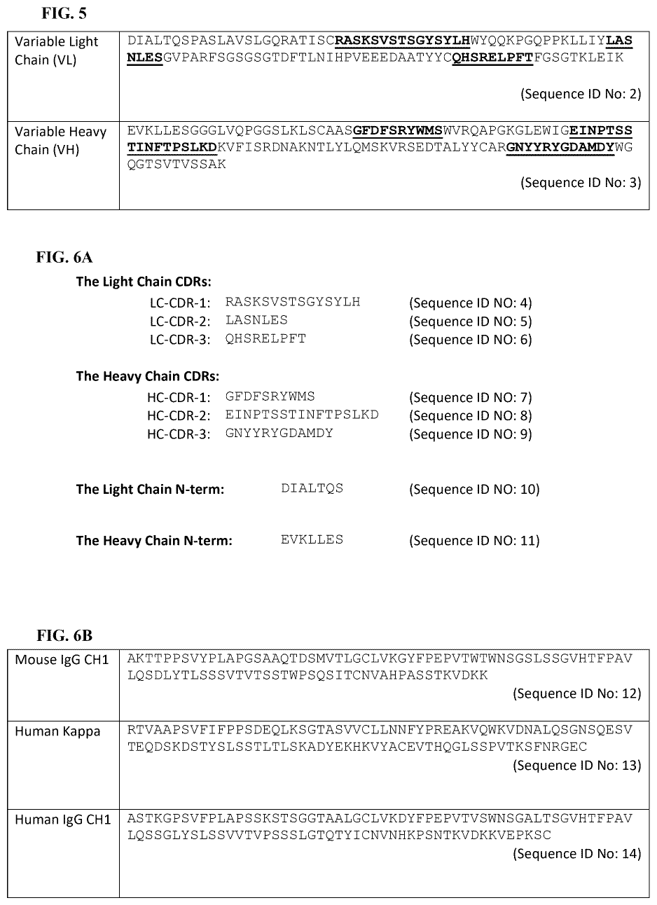

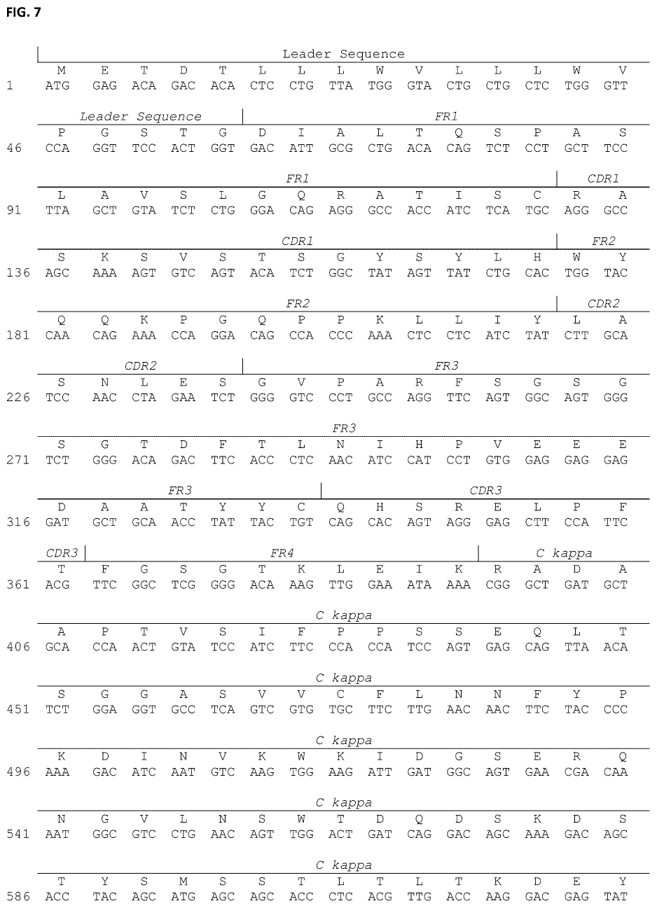

The present invention is also directed to the sequence-related characteristics of the pan-specific anti-CD45 antibody BC8, and specifically to those sequence related characteristics of the N-terminal region and the complementarity determining regions (CDR) of the light chain and the heavy chain of BC8 (see FIGS. 5, 6A, and 6B, respectively). For the light chain, these regions comprise sequences defined by any of: a portion of the N-terminal amino acid sequence as set forth in SEQ ID NO: 10; and the amino acid sequences for the CDR1, CDR2 and CDR3 regions as set forth in SEQ ID NO: 4, SEQ ID NO: 5 and SEQ ID NO: 6, respectively. For the heavy chain, these regions comprises sequences defined by any of: a portion of the N-terminal amino acid sequence as set forth in SEQ ID NO: 11; and the amino acid sequences for the CDR1, CDR2 and CDR3 regions as set forth in SEQ ID NO: 7, SEQ ID NO: 8 and SEQ ID NO: 9, respectively.

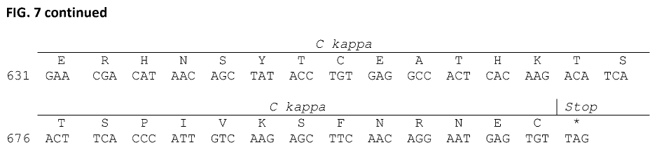

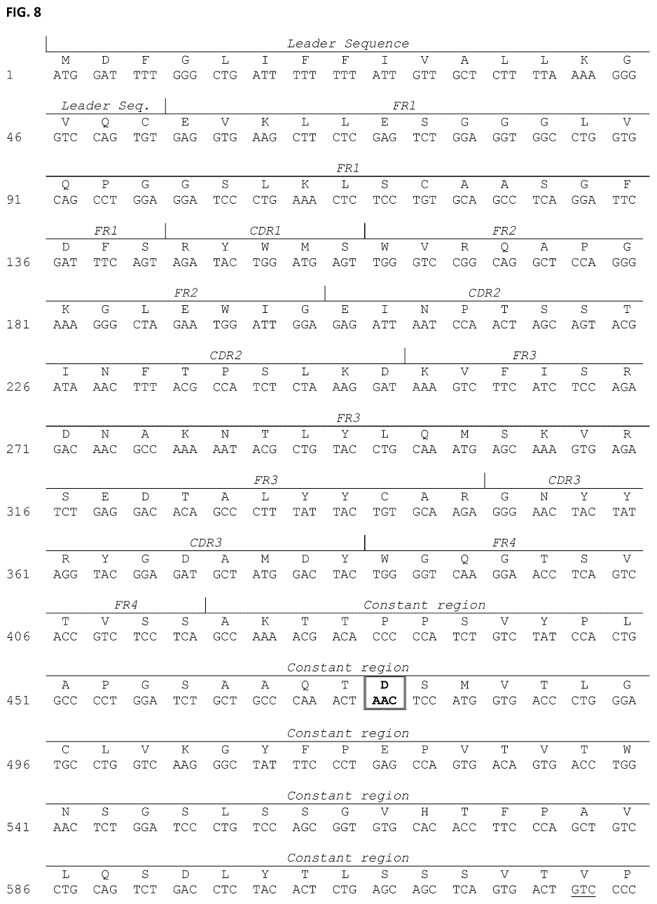

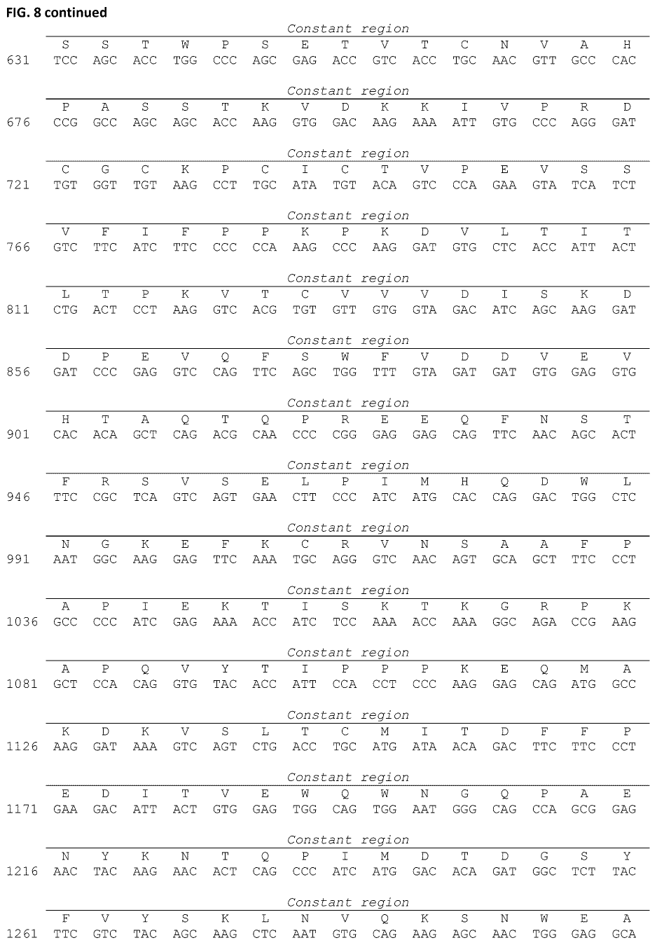

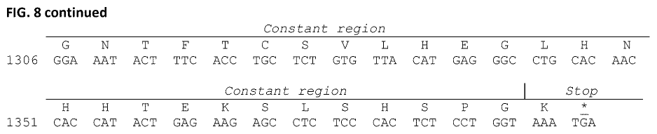

The entire sequence for each of the light and the heavy chains of the anti-CD45 immunoglobulin BC8, as elucidated by RT-PCR-derived cDNA constructs and LC-MS/MS peptide mapping approaches, are also provided in FIGS. 7 and 8 (SEQ ID NO: 12 and 13 for the light chain and SEQ ID NO: 14 and 15 for the heavy chain, for the amino acid and nucleotide sequences, respectively). It is possible that certain isomeric amino acid replacements with exact mass, such as Leu for Ile or vice versa, could be allowed in these sequences. Additionally, certain portions of these sequences may be substituted, such as by related portions from human immunoglobulins to form chimeric immunoglobulins (i.e., chimeric or humanized BC8). Exemplary substitutions include all or portions of the human leader sequence, and/or the conserved regions from human IgG1, IgG2, or IgG4 heavy chains and/or human Kappa light chain.

Accordingly, the present invention includes binding agents that comprise all or portions of any of the protein sequences disclosed in SEQ ID NOS: 2-15, and 17. These binding agents may include antibodies, antibody fragments, and peptides. As such, the invention also relates to antibodies such as BC8, and fragments of an antibody or peptides that may bind to any of the epitopes of CD45 disclosed herein. Exemplary antibody fragments and peptides may comprise sequences and structures defined by, or comprising any of SEQ ID NOS: SEQ ID NOS: 2-15, and 17, or encoded by any portion of the polynucleotide sequences of SEQ ID NOS: 16 and/or 18.

The invention further relates to polynucleotides encoding a binding agent capable of binding to any of the epitopes of CD45 disclosed herein, such as the epitopes comprising amino acids in the cysteine-rich spacer region of CD45. Accordingly, the present invention also includes isolated polynucleotide(s) encoding a monoclonal antibody, antibody fragment, or peptide that binds to amino acids in the cysteine-rich spacer region of CD45, wherein the amino acids in the cysteine-rich spacer region of CD45 comprise any of any of: at least two, three, four, five, or six amino acids in the d1 region of human CD45 (SEQ ID NO: 19); at least two, three, four, five, or six amino acids in the region 228-288 of human CD45 (SEQ ID NO: 20); at least two, three, four, five, or six amino acids in the region 254-286 of human CD45 (SEQ ID NO: 21); at least two, three, four, five, or six of the amino acids V254, N257, E259, N267, N268, H285, N286 of human CD45 (see SEQ ID NO: 1); amino acids V254, N257, E259, N267, H285, N286 of human CD45 (see SEQ ID NO: 1); or amino acids V254, N257, E259, N267, N268, H285, N286 of human CD45 (see SEQ ID NO: 1).

The invention further relates to polypeptide constructs comprising two, three, or four regions of polypeptide structure (i.e., portions of secondary structure) representing the cysteine-rich spacer region of CD45, or a portion thereof, and binding agents that specifically bind thereto. The polypeptide constructs may comprise one or more polypeptide fragments and may further comprise one or more disulfide bonds. Exemplary polypeptide constructs may comprise sequences and structures defined by any of SEQ ID NOS: 19-21. The polypeptide constructs may comprise at least two, three, four, five, or six amino acids from the sequences and structures defined by any of SEQ ID NOS: 19-21.

The polypeptide constructs may comprise a three-dimensional structure that positions at least two, three, four, five, or six of the amino acids V254, N257, E259, N267, N268, H285, N286 of human CD45; or the amino acids V254, N257, E259, N267, H285, N286 of human CD45; or the amino acids V254, N257, E259, N267, N268, H285, N286 of human CD45 within 1-2 Angstroms (Å) of the position of those residues in the native human CD45 protein. As such, the polypeptide constructs may mimic a conformational epitope of the native CD45.

The binding agents disclosed herein may be provided as compositions that include one or more pharmaceutically acceptable salt or diluent. These compositions may be formulated as a patient specific composition, such as at a therapeutically effective dose of one or more of the binding agents disclosed herein, wherein the effective dose is tailored for a specific patient (e.g., based on patient characteristics such as weight, sex, age, disease type and progression, etc.).

As used herein, a patient specific composition may include a radionuclide labeled binding agent, i.e., a radioconjugate, wherein the composition includes both a radionuclide labeled portion and a non-labeled portion. For example, the composition may comprise both an ²²⁵Ac-labeled portion and a non-labeled portion of the binding agent, with the minority being the ²²⁵Ac-labeled portion. The ratio of labeled to non-labeled portions can be adjusted using known methods.

According to certain aspects of the present invention, the binding agent may be provided in a total protein amount of up to 60 mg, such as 5 mg to 45 mg, or a total protein amount of 0.1 mg/kg patient weight to 1.0 mg/kg patient weight, such as 0.2 mg/kg patient weight to 0.6 mg/kg patient weight. The amount of radionuclide-labeled agent provided in the composition may depend on several factors, including the specific identity of the radionuclide.

According to certain aspects of the present invention, the composition may comprise a labeled fraction and an non-labeled fraction of the binding agent, wherein the ratio of labeled: non-labeled may be from about 0.01:10 to 1:10, such as 0.01:5 to 0.1:5, or 0.01:3 to 0.1:3, or 0.01:1 to 0.1:1 labeled: non-labeled. Moreover, the composition may be provided as a single dose composition tailored to a specific patient, wherein the amount of labeled and non-labeled binding agent in the composition may depend on at least a patient weight, age, gender, and/or disease state or health status. See for example administration methods disclosed in International Publication No. WO 2016/187514, incorporated by reference herein in its entirety. According to certain aspects, the radiolabeled antibody or other biologic delivery vehicle may be provided in multiple doses, wherein each dose in the regime may comprise a composition tailored to a specific patient, wherein the amount of labeled and non-labeled antibody or other biologic delivery vehicle in the composition may depend on at least a patient weight, age, gender, and/or disease state or health status.

This inventive combination of a labeled fraction and a non-labeled fraction of the binding agent allows the composition to be tailored to a specific patient, wherein each of the radiation dose and the protein dose of the binding agent are personalized to that patient based on at least one patient specific parameter. As such, each vial of the composition may be made for a specific patient, where the entire content of the vial is delivered to that patient in a single dose. When a treatment regime calls for multiple doses, each dose may be formulated as a patient specific dose in a vial to be administered to the patient as a “single dose” (i.e., full contents of the vial administered at one time). The subsequent dose may be formulated in a similar manner, such that each dose in the regime provides a patient specific dose in a single dose container. One of the advantages of the disclosed composition is that there will be no left-over radiation that would need to be discarded or handled by the medical personnel, e.g., no dilution, or other manipulation to obtain a dose for the patient. When provided in a single dose container, the container is simply placed in-line in an infusion tubing set for infusion to the patient. Moreover, the volume can be standardized so that there is a greatly reduced possibility of medical error (i.e., delivery of an incorrect dose, as the entire volume of the composition is to be administered in one infusion).

According to certain aspects, the binding agent may be labelled with a radioisotope. Examples of radioisotopes include any of ³²P, ²¹¹At, ¹³¹I, ¹³⁷Cs, ⁹⁰Y, ¹⁷⁷Lu, ¹⁸⁶Re, ¹⁸⁸Re, ⁸⁹Sr, ¹⁵³Sm, ²²⁵Ac, ²¹³Bi, ²¹³Po, ²¹²Bi, ²²³Ra, ²²⁷Th, ¹⁴⁹Th, ¹⁶⁴Cu, ²¹²Pb, ⁸⁹Zr, ⁶⁸Ga, and ¹⁰³Pd. Thus, the radiolabeled binding agents (Agent) envisioned in this invention include, without limitation, ³²P-Agent, ²¹¹At-Agent, ¹³¹I-Agent, ¹³⁷Cs-Agent, ⁹⁰Y-Agent, ⁸⁹Sr-Agent, ¹⁵³Sm-Agent, ³²P-Agent, ²²⁵Ac-Agent, ²¹³Bi-Agent, ²¹³Po-Agent, ²¹¹At-Agent, ²¹²Bi-Agent, ²¹³Bi-Agent, ²²³Ra-Agent, ²²⁷Th-Agent, ¹⁴⁹Tb-Agent, ¹³¹I-Agent, ¹³⁷Cs-Agent, ²¹²Pb-Agent, and ¹⁰³Pd-Agent.

The binding agents disclosed herein are able to specifically bind to, and inhibit, the wild type CD45 in vivo. As such, they may be useful as therapeutic agents for the treatment of proliferative diseases and disorders, such as hematological diseases and disorders (i.e., both blood-born and solid cancers).

Additionally, these binding agents may also be useful to effect depletion of lymphocytes, i.e. lymphodepletion, and effect myeloablation, depending on the dose. This depletion method (also referred to herein as a conditioning method) may be useful, for example, for improving the outcome of a subsequent gene-edited cell-based therapy where the depletion of hematopoietic stem cells is desirable.

Moreover, they may be useful for the treatment of non-malignant disorders, i.e., non-cancerous. Thus, according to certain aspects of the present invention, compositions comprising the presently disclosed radiolabeled binding agents may be useful for treating a subject afflicted with a non-cancerous disorder treatable via genetically edited cell therapy, wherein the subject is about to undergo such therapy to treat the disorder. The presently disclosed invention also provides a method for treating a subject afflicted with a non-cancerous disorder treatable via genetically edited cell therapy comprising (i) administering to the subject an amount of a radiolabeled binding agent effective to deplete the subject's hematopoietic stem cells, and (ii) after a suitable time period, performing the therapy on the subject to treat the subject's disorder.

Examples of non-cancerous disorders include, without limitation, hemoglobinopathies (e.g., SCD and β-thalassemia), congenital immunodeficiencies (e.g., SCID and Fanconi's anemia) and viral infections (e.g., HIV infection). According to certain aspects, the disorder is SCD and the therapy is genetically edited β-globin hematopoietic stem cell therapy. The stem cell therapy can be allogenic or autologous, for example. According to certain aspects, the disorder is SCID and the therapy is genetically edited hematopoietic stem cell therapy, wherein the edited gene is the common gamma chain (γc) gene, the adenosine deaminase (ADA) gene and/or the Janus kinase 3 (JAK3) gene. The stem cell therapy can be allogenic or autologous, for example.

Proposed methods by which these binding agents, which include BC8, and antibodies, antibody fragments, peptides, and small molecules that bind to the epitopes of CD45 disclosed herein, may eliminate or deplete CD45-positive cells include antibody-dependent cellular cytotoxicity (ADCC), complement-dependent cytotoxicity (CDC), and apoptosis.

“Antibody-dependent cellular cytotoxicity”, “antibody-dependent cell-mediated cytotoxicity” or “ADCC” is a mechanism for inducing cell death that depends upon the interaction of antibody-coated target cells with effector cells possessing lytic activity, such as natural killer (NK) cells, monocytes, macrophages and neutrophils via Fc gamma receptors (FcγR) expressed on effector cells. For example, NK cells express FcγRIIIa, whereas monocytes express FcγRI, FcγRII and FcvRIIIa. Death of the antibody-coated target cell, such as CD45-expressing cells, occurs as a result of effector cell activity through the secretion of membrane pore-forming proteins and proteases.

“Complement-dependent cytotoxicity”, or “CDC”, refers to a mechanism for inducing cell death in which an Fc effector domain of a target-bound antibody binds and activates complement component C1q, which in turn activates the complement cascade leading to target cell death. Activation of complement may also result in deposition of complement components on the target cell surface that facilitate ADCC by binding complement receptors (e.g., CR3) on leukocytes.

“Apoptosis” refers to a mechanism of programmed cell death wherein antibody binding to the target cell disrupts integral cell signaling pathways and results in cell self-destruction.

To assess ADCC activity of an antibody or binding agent that specifically binds CD45, the agent may be added to CD45-expressing cells in combination with immune effector cells, which may be activated by the antigen-antibody complexes resulting in cytolysis of the CD45-expressing cells. Cytolysis is generally detected by the release of a label (e.g. radioactive substrates, fluorescent dyes or natural intracellular proteins) from the lysed cells. Exemplary effector cells for such assays include peripheral blood mononuclear cells (PBMC) and NK cells.

In an exemplary assay for ADCC, CD45-expressing cells may be labeled with 51Cr and washed extensively. Anti-CD45 antibodies or binding agents may be added to the CD45-expressing cells at various concentrations, and the assay started by adding effector cells (NK cells from peripheral blood mononuclear cells, for example). After incubation for various time intervals at 37° C., assays are stopped by centrifugation and 51Cr release from lysed cells is measured in a scintillation counter. Percentage of cellular cytotoxicity may be calculated as % maximal lysis which may be induced by adding 3% perchloric acid to the CD45-expressing cells.

In an exemplary assay for cytotoxicity, tetrazolium salt is added to CD45-expressing cells treated with various amounts of anti-CD45 antibodies. In living mitochondria, the XTT is reduced to an orange product by mitochondrial dehydrogenase and transferred to the cell surface. The orange product can be optically quantified and reflects the number of living cells. Alternatively, esterases from living cells are known to hydrolyze the colorless calcenin into as fluorescent molecule. The fluorescence can be measured and quantified and reflects the number of living cells in the sample. The total amount of dead cells may be measured using propidium iodide, which is excluded from live cells by intact membranes. The fluorescence due to the propidium iodide in dead cells may be quantified by flow-cytometry.

In order to assess CDC, complement protein, such as C1q, may need to be included in an assay for cytotoxicity. Measurement of apoptosis induction does not require addition of NK cells or complement protein in an assay for cytotoxicity.

Accordingly, the present invention solves an unmet need in the art by providing an unexpectedly superior way to inhibit CD45 expressing cells in a targeted manner using the disclosed binding agents. When provided as antibody fragments, peptides, or small molecules, these agents may also provide the additional benefit of improved clearance rates from the subject, thus reducing the negative side effects of standard immunotherapies.

The present invention is thus also directed to methods for treating proliferative diseases and disorders, such as hematological diseases and disorders. Additionally, these binding agents may be useful to effect depletion of lymphocytes, i.e. lymphodepletion, and effect myeloablation. For example, the presently disclosed binding agents may be used to lymphodeplete a subject prior to a cell-based therapy like CAR T-cell therapy or TCR cell therapy. When the binding agent is radiolabeled, such as with ¹³¹I or ²²⁵Ac, surprisingly low doses may be effective, thus avoiding certain adverse effects caused by less specific agents like chemotherapeutics. Also, using this approach, at least some types of CD45+ immune cells, such as neutrophils, may also surprisingly avoid significant depletion.

This lymphodepletion method is useful, for example, for improving the outcome of a subsequent therapy wherein the depletion of lymphocytes is desirable. According to certain preferred aspects of this method, the subject is afflicted with cancer and is about to undergo adoptive cell therapy to treat the cancer (e.g., hematological malignancy or solid cancer). Adoptive cell therapies are known, and include, for example, CAR T-cell therapy (e.g., autologous cell therapy and allogeneic cell therapy). Preferred are CAR T-cell therapies for treating hematologic malignancies such as ALL, AMI, and CLL. Examples of approved CAR T-cell therapies include, without limitation, KYMRIAH® (tisagenlecleucel) for treating NHL and DLBCL, and YESCARTA® (axicabtagene ciloleucel) for treating NHL.

These presently disclosed methods may improve treatment outcomes for hematological malignancies including solid tumors, and/or may lessen side effects associated with the adoptive cell therapies, such as the CAR T-cell therapies KYMRIAH®) and/or YESCARTA®). For example, side effects of adoptive cell therapies include neurotoxicity, cytokine release syndrome (CRS), hypogammaglobulinemia, cytopenias, capillary leak syndrome (CLS), macrophage activation syndrome (MAS), tumor lysis syndrome (TLS), and combinations thereof. Moreover, the presently disclosed methods may prolong persistence of the population of cells expressing the CAR/TCR or the TIL when compared to a method absent administration of the radiolabeled anti-CD45 antibody.

The present invention further provides methods for targeted lymphodepletion of immune suppressor cells such as regulatory T (T-reg) cells and myeloid-derived suppressor cells (MDSC). Both cells types (i.e., T-regs and MDSC) can dampen the activation and efficacy of CAR T-cell therapies. Moreover, the present invention also provides methods for targeted lymphodepletion of immune suppressor cells such as monocytes and tumor-associated macrophages (TAMs) that have been implicated in cytokine release contributing to toxicities such as cytokine release syndrome (CRS) and neurotoxicity associated with CAR T-cells.

Tumors, both solid and liquid have evolved methods to hijack and/or evade the immune system as a means to perpetuate and thrive. This has been called the hostile tumor immune microenvironment (TME). A classical and relevant example is the up-regulated expression of the ligand PD-L1 on the tumor cell surface to bind PD1 on the surface of T cells, leading to down-modulation of immune cell activation. Interestingly, although blockade of this mechanism has led to remarkable response rates and durable survival in several different types of cancer, most patients do not respond to this form of therapy (i.e., anti-PD1/PD-L1), implying that immune evasion in the tumor micro-environment is multi-faceted and complex. To this end, the tumor, in part through oncogenic expression, signaling, and cytokine production, can confer challenges on the immune system, hindering the mounting of an effective anti-tumor response. This can lead to an environment characterized by oxidative stress, nutrient depletion, an acidic pH, and hypoxia. Further, the presence of these suppressive immune cells (T-regs and MDSC), and tumor-associated macrophages (TAM) can effectively blunt immune cell activation through direct contact or release of suppressive soluble factors and cytokines.

While a patient's endogenous immune system may encounter such an environment and lead to a compromised anti-tumor immune response, adoptive cell therapies such as CAR T-cell therapy may also be susceptible to these immune suppressive mechanisms, restricting the ability of these novel cell therapies to mount an effective response to the tumor.

The tumor immune microenvironment has also been implicated in the two primary adverse events associated with CAR-T administration, namely cytokine release syndrome (CRS) and neurotoxicity. Recent preclinical studies have shown that cytokine release leading to CRS or neurotoxicity is due to activated macrophages following recruitment to the site of CAR-T and tumor cells. Mouse study result. (Giavadris, et al., 2018, Nat. Med., 24:731) documented that macrophages secrete IL-1 or IL-6 following recruitment and activation by CAR-T cells at the tumor site.

Conditioning has been shown to improve the immune homeostatic environment to enable successful ACT or CAR-T engraftment and expansion in vivo following infusion. However, the use of cytotoxic non-specific chemotherapy can elicit off-target toxicities and has been identified as a risk factor in CRS and neurotoxicity following CAR-T administration (Hay, et al., 2016). Interestingly, most CAR-T programs exploit the use of the combination of fludarabine and cyclophosphamide (flu/cy) as a conditioning regimen prior to CAR-T. These drugs are often administered 2-7 days prior to ACT infusion, using 2-5 day course of therapy.

The targeted therapy for conditioning of the present invention offers an improved strategy for enhancing outcomes with CAR-T. In the invention described herein, not only may lymphocytes be targeted for depletion, but also those immune cell types implicated in mediating a hostile tumor immune microenvironment, and those implicated in CAR-T adverse events such as CRS and neurotoxicity. The present invention targets normal immune cells including T-regs, MDSCs, TAMs, and activated macrophages secreting IL-1 and/or IL-6. In doing so, the invention may have a dramatic improvement in CAR-T outcomes and safety.

Furthermore, the invention will target, primarily in hematopoietic tumors, patient cancer cells to reduce tumor burden and increase the probability of CAR-T anti-tumor response. More specifically, the invention provides a therapeutic strategy targeting the specific epitopes of CD45, which is found on all normal nucleated immune cells with the exception of red blood cells and platelets. CD45 is also expressed on most lymphoid and leukemic tumor cells. While naked antibodies have shown some impact on reducing immune cell populations, the binding agents of the presently disclosed invention, either labelled or radiolabeled, will affect a more pronounced and sustained suppression of immune cells implicated in modulating CAR-T responses, consistent with, but in a targeted manner, to external beam radiation. In this way, the radiation is targeted and impactful on the CD45 cell populations while sparing normal tissues. More specifically, the binding agents may be provided as a single dose at a level sufficiently effective to deplete circulating immune cells within the spleen, lymph nodes, and peripheral blood, but limited in impact on hematopoietic stem cells in the bone marrow. Importantly, in addition to lymphocyte depletion, macrophages, MDSCs and T-regs will be depleted to improve the activation and response to CAR-T therapy and mitigate adverse events CRS and neurotoxicity.

The binding agents of the present invention may be administered intravenously, intramuscularly, or subcutaneously to a patient. Exemplary administration amounts and rates for the compositions may be as substantially described in WO 2016/187514, incorporated by reference herein. Additionally, when provided as small molecules, the binding agents may be administered as oral formulations (e.g., liquid or solid forms).

According to certain aspects, the binding agents may be radiolabeled, such as with ¹³¹I. Examples of effective amounts include, without limitation, from 50 mCi to 100 mCi, from 50 mCi to 150 mCi, from 50 mCi to 200 mCi, from 60 mCi to 140 mCi, from 70 mCi to 130 mCi, from 80 mCi to 120 mCi, from 90 mCi to 110 mCi, from 100 mCi to 150 mCi, 50 mCi, 60 mCi, 70 mCi, 80 mCi, 90 mCi, 100 mCi, 110 mCi, 120 mCi, 130 mCi, 140 mCi, 150 mCi, or 200 mCi. According to certain aspects, the effective amount is from 10 mCi to 120 mCi, from 20 mCi to 110 mCi, from 25 mCi to 100 mCi, from 30 mCi to 100 mCi, from 40 mCi to 100 mCi, from 50 mCi to 100 mCi, or from 75 mCi to 100 mCi.

According to certain aspects, the binding agents may be radiolabeled, such as with 225Ac. Examples of effective amounts include, without limitation, from 0.05 μCi/kg to 5.0 μCi/kg, such as from 0.1 μCi/kg to 0.2 μCi/kg, from 0.2 μCi/kg to 0.3 μCi/kg, from 0.3 μCi/kg to 0.4 μCi/kg, from 0.4 μCi/kg to 0.5 μCi/kg, from 0.5 μCi/kg to 0.6 μCi/kg, from 0.6 μCi/kg to 0.7 μCi/kg, from 0.7 μCi/kg to 0.8μ Ci/kg, from 0.8μ Ci/kg to 0.9μ Ci/kg, from 0.9μ Ci/kg to 1.0 μCi/kg, from 1.0 μCi/kg to 1.5 μCi/kg, from 1.5 μCi/kg to 2.0 μCi/kg, from 2.0 μCi/kg to 2.5 μCi/kg, from 2.5 μCi/kg to 3.0 μCi/kg, from 3.0 μCi/kg to 3.5 μCi/kg, from 3.5 μCi/kg to 4.0 μCi/kg, from 4.0 μCi/kg to 4.5 μCi/kg, or from 4.5 μCi/kg to 5.0 μCi/kg.

The effective amount of the binding agent may be provided as a single dose. A majority of the binding agent administered to a subject typically consists of non-labeled binding agent, with the minority being the labeled binding agent. The ratio of labeled to non-labeled binding agent can be adjusted using known methods. Thus, accordingly to certain aspects of the present invention, the binding agent may be provided in a total protein amount of up to 100 mg, such as less than 60 mg, or from 5 mg to 45 mg, or a total protein amount of between 0.1 mg/kg patient weight to 1.0 mg/kg patient weight, such as from 0.2 mg/kg patient weight to 0.6 mg/kg patient weight.

According to certain aspects of the present invention, the radiolabeled binding agent may comprise a labeled fraction and an unlabeled fraction, wherein the ratio of labeled: unlabeled may be from about 0.01:10 to 1:1, such as 0.1:10 to 1:1 labeled: unlabeled. Moreover, the radiolabeled anti-CD45 antibody may be provided as a single dose composition tailored to a specific patient, wherein the amount of labeled and unlabeled binding agent in the composition may depend on at least a patient weight, age, and/or disease state or health status.

The binding agents of the present invention may also be administered in combination therapy, i.e., combined with other therapeutic agents relevant for the disease or condition to be treated. Such administration may be simultaneous, separate or sequential. For simultaneous administration, the agents may be administered as one compositions or as separate compositions, as appropriate.

According to certain aspects of the present invention, the pharmaceutical composition may include one or more therapeutic agents. Exemplary therapeutic agents include a chemotherapeutic agent, an anti-inflammatory agent, an immunosuppressive, an immunomodulatory agent, or a combination thereof.

Therapeutic agents may be administered according to any standard dose regime known in the field. When therapeutic agents are included in the composition of the present invention, they may be included at concentrations in the range of 1 to 500 mg/m², the amounts being calculated as a function of patient surface area (m²). For example, exemplary doses of paclitaxel may include 15 mg/m² to 275 mg/m², exemplary doses of docetaxel may include 60 mg/m² to 100 mg/m², exemplary doses of epothilone may include 10 mg/m² to 20 mg/m², and an exemplary dose of calicheamicin may include 1 mg/m² to 10 mg/m². While exemplary doses are listed herein, such are only provided for reference and are not intended to limit the dose ranges of the drug agents of the presently disclosed invention.

Thus, according to one aspect, the pharmaceutical composition may include at least one chemotherapeutic agent. Exemplary chemotherapeutic agents include an antimetabolite, such as methotrexate, 6-mercaptopurine, 6-thioguanine, cytarabine, fludarabine, 5-fluorouracil, decarbazine, hydroxyurea, asparaginase, gemcitabine, cladribine and similar agents.

Exemplary chemotherapeutic agents include an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin, and similar agents.

Exemplary chemotherapeutic agents include an antibiotic, such as dactinomycin (formerly actinomycin), bleomycin, calicheamicin, daunorubicin (formerly daunomycin), doxorubicin, idarubicin, mithramycin, mitomycin, mitoxantrone, plicamycin, anthramycin (AMC) and similar agents.

Exemplary chemotherapeutic agents include anti-mitotic agent, such as taxanes, for instance docetaxel, and paclitaxel, and vinca alkaloids, for instance vindesine, vincristine, vinblastine, and vinorelbine.

Exemplary chemotherapeutic agents include a topoisomerase inhibitor, such as topotecan.

Exemplary chemotherapeutic agents include a growth factor inhibitor, such as an inhibitor of ErbB1 (EGFR) (such as gefitinib (Iressa®), cetuximab (Erbitux®), erlotinib (Tarceva®)), HuMax-EGFr (2F8 disclosed in WO 2002/100348) and similar agents), an inhibitor of ErbB2 (Her2/neu) (such as trastuzumab (Herceptin®)) and similar agents) and similar agents. In one embodiment, such a growth factor inhibitor may be a farnesyl transferase Inhibitor, such as SCH-66336 and R115777. In one embodiment, such a growth factor inhibitor may be a vascular endothelial growth factor (VEGF) inhibitor, such as bevacizumab (Avastin®).

Exemplary chemotherapeutic agents include a tyrosine kinase inhibitor, such as imatinib (Glivec, Gleevec ST1571), lapatinib, PTK787/ZK222584 and similar agents.

Exemplary chemotherapeutic agents include a histone deacetylase inhibitor. Examples of such histone deacetylase inhibitors include hydroxamic acid-based hybrid polar compounds, such as SAHA (suberoylanilide hydroxamic acid).

Exemplary chemotherapeutic agents include a P38a MAP kinase inhibitor, such as SCIO-469.

Exemplary chemotherapeutic agents include inhibitors of angiogenesis, neovascularization, and/or other vascularization. Examples of such inhibitors include urokinase inhibitors, matrix metalloprotease inhibitors (such as marimastat, neovastat, BAY 12-9566, AG 3340, BMS-275291 and similar agents), inhibitors of endothelial cell migration and proliferation (such as TNP-470, squalamine, 2-methoxyestradiol, combretastatins, endostatin, angiostatin, penicillamine, SCH66336 (Schering-Plough Corp, Madison, N.J.), R115777 (Janssen Pharmaceutica, Inc, Titusville, N.J.) and similar agents), antagonists of angiogenic growth factors (such as such as ZD6474, SU6668, antibodies against angiogenic agents and/or their receptors (such as VEGF, bFGF, and angiopoietin-1), thalidomide (Thalomid®)), thalidomide analogs (such as CC-5013 (lenalidomide, Revlimid™) and CC4047 (Actimid™), Sugen 5416, SU5402, antiangiogenic ribozyme (such as angiozyme), interferon a (such as interferon α2α), suramin and similar agents), VEGF-R kinase inhibitors and other anti-angiogenic tyrosine kinase inhibitors (such as SU011248), inhibitors of endothelial-specific integrin/survival signaling (such as vitaxin and similar agents), copper antagonists/chelators (such as tetrathiomolybdate, captopril and similar agents), carboxyamido-triazole (CAI), ABT-627, CM101, interleukin-12 (IL-12), IM862, PNU145156E as well as nucleotide molecules inhibiting angiogenesis (such as antisense-VEGF-cDNA, cDNA coding for angiostatin, cDNA coding for p53 and cDNA coding for deficient VEGF receptor-2) and similar agents.

Other examples of such inhibitors of angiogenesis, neovascularization, and/or other vascularization are anti-angiogenic heparin derivatives and related molecules (e.g., heperinase III), temozolomide, NK4, macrophage migration inhibitory factor (MIF), cyclooxygenase-2 inhibitors, inhibitors of hypoxia-inducible factor 1, anti-angiogenic soy isoflavones, oltipraz, fumagillin and analogs thereof, somatostatin analogues, pentosan polysulfate, tecogalan sodium, dalteparin, tumstatin, thrombospondin, NM-3, combrestatin, canstatin, avastatin, antibodies against other relevant targets (such as anti-alpha-v/beta-3 integrin and anti-kininostatin mAbs) and similar agents.

Exemplary chemotherapeutic agents include thalidomide (Thalomid®)), thalidomide analogs (such as CC-5013 (lenalidomide, Revlimid™) and/or CC4047 (Actimid™).

Exemplary chemotherapeutic agents may include additional antibody therapeutics, or drugs such as a proteasome inhibitor, such as bortezomib (Velcade®)), a corticosteroid, such as prednisone, prednisolone, dexamethasone, etc., a bisphosphonate. Examples of potentially suitable biphosphonates are pamidronate (Aredia®)), zoledronic acid (Zometa®), clodronate (Bonefos®), risendronate (Actonel®)), ibandronate (Boniva®), etidronate (Didronel®), alendronate (Fosamax®), tiludronate (Skelid®)), incadronate (Yamanouchi Pharmaceutical) and minodronate (YM529, Yamanouchi).

Exemplary chemotherapeutic agents include a colony stimulating factor. Examples of suitable colony stimulating factors are granulocyte-colony stimulating factors (G-CSF), such as filgrastim (Neupogen®)) and pegfilgrastim (Neulasta®)), and granulocyte macrophage-colony stimulating factors (GM-CSF) such as sargramostim (Leukine®)).

Exemplary chemotherapeutic agents include an erythropoietic agent. Examples of suitable erythropoietic agents are erythropoietin (EPO), such as epoetin alfa (for instance Procrit®), Epogen®), and Eprex®)) and epoetin beta (for instance NeoRecormon®) and erythropoiesis-stimulating proteins (for instance Aranesp®)).

Exemplary chemotherapeutic agents include an anti-anergic agents (for instance small molecule compounds, proteins, glycoproteins, or antibodies that break tolerance to tumor and cancer antigens).

Exemplary chemotherapeutic agents include a virus, viral proteins, and the like. Replication-deficient viruses, that generally are capable of one or only a few rounds of replication in vivo, and that are targeted to tumor cells, may for instance be useful components of such compositions and methods. Such viral agents may comprise or be associated with nucleic acids encoding immunostimulants, such as GM-CSF and/or IL-2. Both naturally oncolytic and such recombinant oncolytic viruses (for instance HSV-1 viruses, reoviruses, replication-deficient and replication-sensitive adenovirus, etc.) may be useful components of such methods and compositions.

According to another aspect, the pharmaceutical composition may include an anti-inflammatory agent may be selected from a steroidal drug and a NSAID (nonsteroidal anti-inflammatory drug). Other anti-inflammatory agents may be selected from aspirin and other salicylates, Cox-2 inhibitors (such as rofecoxib and celecoxib), NSAIDs (such as ibuprofen, fenoprofen, naproxen, sulindac, diclofenac, piroxicam, ketoprofen, diflunisal, nabumetone, etodolac, oxaprozin, and indomethacin), anti-IL6R antibodies, anti-IL8 antibodies, anti-IL15 antibodies, anti-IL15R antibodies, anti-CD4 antibodies, anti-CD11a antibodies (e.g., efalizumab), anti-alpha4/beta-1 integrin (VLA4) antibodies (e.g. natalizumab), CTLA4-1 g for the treatment of inflammatory diseases, prednisolone, prednisone, disease modifying antirheumatic drugs (DMARDs) such as methotrexate, hydroxychloroquine, sulfasalazine, pyrimidine synthesis inhibitors (such as leflunomide), IL-1 receptor blocking agents (such as anakinra), TNF-α blocking agents (such as etanercept, infliximab, and adalimumab) and similar agents.

According to another aspect, the pharmaceutical composition may include at least one immunosuppressive and/or immunomodulatory agent to a subject in need thereof. Examples of an immunosuppressive and/or immunomodulatory agent include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-α and similar agents.

Additional immunosuppressive and/or immunomodulatory agents may be selected from immunosuppressive antibodies, such as antibodies binding to p75 of the II-2 receptor, or antibodies binding to for instance MHC, CD2, CD3, CD4, CD7, CD28, B7, CD40, CD45, IFNγ, TNF-α, IL-4, IL-5, IL-6R, IL-6; IGF, IGFR1, IL-7, IL-8, IL-10, CD11a, or CD58, or antibodies binding to their ligands. Further additional immunosuppressive and/or immunomodulatory agents may be selected from soluble IL-15R, IL-10, B7 molecules (B7-1, B7-2, variants thereof, and fragments thereof, ICOS, and OX40, an inhibitor of a negative T cell regulator (such as an antibody against CTLA4) and similar agents.

According to certain aspects of the present invention, the one or more therapeutic agents comprises an antimyeloma agent. Exemplary antimyeloma agents include dexamethasone, melphalan, doxorubicin, bortezomib, lenalidomide, prednisone, carmustine, etoposide, cisplatin, vincristine, cyclophosphamide, and thalidomide, several of which are indicated above as chemotherapeutic agents, anti-inflammatory agents, or immunosuppressive agents.

The binding agents of the present invention may be used to treat a hematological malignancy, or to inhibit growth and/or proliferation of a cell expressing CD45, or to treat a disease or disorder involving cells expressing CD45. As such, the present invention provides methods for treating a subject having a hematological malignancy, for inhibiting growth and/or proliferation of a cell expressing CD45, and for treating a disease or disorder involving cells expressing CD45, wherein the methods comprise administering to a subject the pharmaceutical composition detailed hereinabove.

According to certain aspects of the present invention, the hematological malignancy is multiple myeloma. According to certain aspects of the invention the cell expressing CD45 is a multiple myeloma cell. According to certain aspects of the invention, the disease or disorder may be multiple myeloma.

According to certain aspects of the invention the cell expressing CD45 are CD45-expressing cancer cells or CD45-expressing T-cells, B-cells, NK cells, or plasma cells. According to certain aspects of the invention the cell expressing CD45 comprise solid tumor cells or hematological malignancy cells. Exemplary hematological malignancy cells comprise multiple myeloma cells, acute lymphocytic leukemia cells, acute myeloid leukemia cells, chronic lymphocytic leukemia cells, chronic myeloid leukemia cells, Hodgkin's lymphoma cells, non-Hodgkin's lymphoma cells, T-LGL leukemia cells, NK cell leukemia cells, or hairy cell leukemia cells.

As indicated above, the pharmaceutical composition may be administered either alone or in combination with one or more additional therapeutic agents. The pharmaceutical composition may comprise the one or more additional therapeutic agents. The pharmaceutical composition may be administered in a dosage regime comprising at least one dose.

According to certain aspects of the present invention, the therapeutically effective dose of the binding agents may be 0.1 ug/kg to 1 mg/kg, such as 1 ug/kg to 1 mg/kg, or 10 ug/kg to 1 mg/kg, or 100 ug/kg to 1 mg/kg, or 0.1 ug/kg to 100 μg/kg, or 0.1 ug/kg to 50 ug/kg, or 0.1 ug/kg to 10 ug/kg, or 0.1 ug/kg to 40 ug/kg, or 1 ug/kg to 40 ug/kg.

These protein doses may include a radiation dose of 0.1 uCi/kg to 5 uCi/kg, such as 0.1 uCi/kg to 4 uCi/kg, or 0.1 uCi/kg to 3 uCi/kg, or 0.1 uCi/kg to 2 uCi/kg, or 0.1 uCi/kg to 1 uCi/kg, or 0.2 uCi/kg to 5 uCi/kg, or 0.5 uCi/kg to 5 uCi/kg. Alternatively, there protein doses may include a radiation dose of 5 mCi to 200 mCi, such 10 mCi to 150 mCi, or 20 mCi to 125 mCi.

The therapeutically effective dose of the radiolabeled binding agents may be administered in a single dose, or as two equal fractionated doses, wherein a second dose may be administered 1 day to 10 days, such as 3-8 days or 4-7 days or 5-8 days, after the first dose.

According to one aspect, the present invention provides methods for treating a cancer in a subject, wherein the method comprises administering to the subject a therapeutically effective amount of the pharmaceutical compositions detailed hereinabove. Without wishing to be bound by any particular theory, the immunomodulatory effects observed with the binding agents described herein may be efficacious in treatment of solid tumors. Thus, the invention also provides for a method of treating a patient having a solid tumor, comprising administering to the patient in need thereof a therapeutically effective amount of a binding agent that specifically binds CD45 for a time sufficient to treat the solid tumor.

According to certain aspects, the present invention provides articles of manufacture. For example, an article of manufacture according to aspects of the present invention may comprise (a) a binding agent specific for an epitope of CD45, and (b) a label instructing a user to administer to a subject an amount of the binding agent effective to treat a disease or disorder involving cells expressing CD45. The binding agent may include any of the binding agents discussed herein and may be labelled with any of the radionuclides discussed herein. According to one aspect of the invention, the article of manufacture may comprise a binding agent wherein at least a portion of the agent is labeled with any of the radioisotopes discloses herein, such as ²²⁵Ac or ¹³¹I. According to further aspects of the article of manufacture, the amount of the binding agent effective to treat the disease or disorder involving cells expressing CD45 comprises 10 μCi to 600 μCi of the ²²⁵Ac-labelled binding agent, such as 10 μCi to 400 μCi of the ²²⁵Ac-labelled binding agent, or 10 μCi to 200 μCi of the 225Ac-labelled binding agent.

According to certain aspects of the present invention, the article of manufacture may comprise (a) a pharmaceutical composition according to any of the aspects discussed herein, and (b) a label instructing a user to administer to a subject an amount of the binding agent effective to treat a disease or disorder involving cells expressing CD45. For example, a pharmaceutical composition may comprise a radiolabeled fraction of a binding agent against an epitope of CD45, and an unlabeled fraction of the same or a different binding agent against the epitope of CD45. The pharmaceutical composition may be provided in a patient specific form, such as at a therapeutically effective dose comprising an amount of radioactivity and a protein concentration that are tailored for a specific patient (e.g., based on patient characteristics such as weight, sex, age, disease type and progression, etc.). According to certain aspects, the therapeutically effective dose may comprise radiolabeled binding agent at 0.1 ug/kg to 1 mg/kg, such as 1 ug/kg to 1 mg/kg, or 10 ug/kg to 1 mg/kg, or 100 ug/kg to 1 mg/kg, or 0.1 ug/kg to 100 ug/kg, or 0.1 ug/kg to 50 ug/kg, or 0.1 ug/kg to 10 ug/kg, or 0.1 ug/kg to 40 ug/kg, or 1 ug/kg to 40 ug/kg; having a radioactive dose of 0.1 uCi/kg to 5 uCi/kg, such as 0.1 uCi/kg to 4 uCi/kg, or 0.1 uCi/kg to 3 uCi/kg, or 0.1 uCi/kg to 2 uCi/kg, or 0.1 uCi/kg to 1 uCi/kg, or 0.2 uCi/kg to 5 uCi/kg, or 0.5 uCi/kg to 5 uCi/kg.

According to certain aspects, the present invention provides a pharmaceutical composition useful for treatment of a disease or disorder involving cells expressing CD45. The composition may comprise 1 to 50 wt. % of a binding agent against CD45 labeled with ¹³¹I or ²²⁵Ac; 50 to 99 wt. % of the monoclonal antibody against CD45 that is unlabeled; and a pharmaceutically acceptable carrier. According to yet further aspects of the pharmaceutical composition, the radiolabeled monoclonal antibody may be ¹³¹I-binding agent or ²²⁵Ac-binding agent, and the unlabeled monoclonal antibody may be the same as the labelled binding agent or may be different.

### Aspects of the Invention

The following aspects are disclosed in this application:

Aspect 1: An isolated polynucleotide encoding a monoclonal antibody, antibody fragment, or peptide that binds to amino acids in the cysteine-rich spacer region of CD45.

Aspect 2: The isolated polynucleotide according to aspect 1, wherein the amino acids in the cysteine-rich spacer region of CD45 comprise any of: at least two amino acids in the d1 region of human CD45 (SEQ ID NO: 19); at least two amino acids in the region 228-288 of human CD45 (SEQ ID NO: 20); at least two amino acids in the region 254-286 of human CD45 (SEQ ID NO: 21); at least two of amino acids V254, N257, E259, N267, N268, H285, N286 of human CD45 (see SEQ ID NO: 1); amino acids V254, N257, E259, N267, H285, N286 of human CD45 (see SEQ ID NO: 1); and amino acids V254, N257, E259, N267, N268, H285, N286 of human CD45 (see SEQ ID NO: 1).

Aspect 3: A binding agent capable of binding an epitope of CD45, wherein the binding agent is an antibody, antibody fragment, peptide, or small molecule, and the epitope comprises amino acids in the cysteine-rich spacer region of CD45.

Aspect 4: The binding agent according to aspect 3, wherein the epitope comprises any of: at least two amino acids in the d1 region of human CD45 (SEQ ID NO: 19); at least two amino acids in the region 228-288 of human CD45 (SEQ ID NO: 20); at least two amino acids in the region 254-286 of human CD45 (SEQ ID NO: 21); at least two of amino acids V254, N257, E259, N267, N268, H285, N286 of human CD45 (see SEQ ID NO: 1); amino acids V254, N257, E259, N267, H285, N286 of human CD45 (see SEQ ID NO: 1); and amino acids V254, N257, E259, N267, N268, H285, N286 of human CD45 (see SEQ ID NO: 1).

Aspect 5: An epitope of CD45, the epitope comprising any of: at least two amino acids in the d1 region of human CD45 (SEQ ID NO: 19); at least two amino acids in the region 228-288 of human CD45 (SEQ ID NO: 20); at least two amino acids in the region 254-286 of human CD45 (SEQ ID NO: 21); at least two of amino acids V254, N257, E259, N267, N268, H285, N286 of human CD45 (see SEQ ID NO: 1); amino acids V254, N257, E259, N267, H285, N286 of human CD45 (see SEQ ID NO: 1); and amino acids V254, N257, E259, N267, N268, H285, N286 of human CD45 (see SEQ ID NO: 1).

Aspect 6: A conformational epitope of CD45, the epitope comprising a three-dimensional structure defined by a portion of the cysteine-rich spacer region of CD45.

Aspect 7: The epitope according to aspect 6, comprising at least one polypeptide fragment having any of: at least two amino acids in the d1 region of human CD45 (SEQ ID NO: 19); at least two amino acids in the region 228-288 of human CD45 (SEQ ID NO: 20); at least two amino acids in the region 254-286 of human CD45 (SEQ ID NO: 21); at least two of amino acids V254, N257, E259, N267, N268, H285, N286 of human CD45 (see SEQ ID NO: 1); amino acids V254, N257, E259, N267, H285, N286 of human CD45 (see SEQ ID NO: 1); and amino acids V254, N257, E259, N267, N268, H285, N286 of human CD45 (see SEQ ID NO: 1).

Aspect 8: Use of the epitope of aspects 5 or 6 to produce a binding agent, wherein the binding agent is an antibody, antibody fragment, peptide, or small molecule capable of specifically binding the epitope.

Aspect 9: The use according to aspect 8, wherein the binding agent inhibits CD45 activity in vivo.

Aspect 10: A method for treating a subject having a proliferative disease, the method comprising: administering to the subject an effective amount of any of the binding agents according to aspects 3 or 4.

Aspect 11: A method for treating a disease or disorder involving cells expressing CD45, the method comprising: administering to the subject an effective amount of any of the binding agents according to aspects 3 or 4.

Aspect 12: A method for inhibiting proliferation of a cell expressing CD45, the method comprising: administering to the subject an effective amount of any of the binding agents according to aspects 3 or 4.

Aspect 13: The method according to any one of aspects 10-12, wherein the binding agent is at least partially labelled with a radiolabel selected from ³²p, ²¹¹At, ¹³¹I, ¹³⁷Cs, ⁹⁰Y, ¹⁷⁷Lu, ¹⁸⁶Re, ¹⁸⁸Re, ⁸⁹Sr, ¹⁵³Sm, ²²⁵Ac, ²¹³Bi, ²¹³Po, ²¹²Bi, ²²³Ra, ²²⁷Th, ¹⁴⁹Tb, ¹⁶⁴Cu, ²¹²Pb, ⁸⁹Zr, ⁶⁸Ga, and ¹⁰³Pd, or a combination thereof.

Aspect 14: The method according to aspect 13, wherein the binding agent comprises ¹³¹I or ²²⁵Ac, and the effective amount of comprises a dose of 0.1 to 10 μCi/kg body weight of the subject, or 0.2 to 8 μCi/kg body weight of the subject, or 0.5 to 4 μCi/kg subject body weight.

Aspect 15: The method according to any one of aspects 10-12, further comprising: administering to the subject an effective amount of a second therapeutic agent.

Aspects 16: The method according to any one of aspects 10-12, further comprising: transplanting allogeneic stem cells to the subject after administration of the binding agent, wherein the effective amount of the binding agent induced myeloablation, and wherein the transplantation is performed 8 to 20 days after the administration of the binding agent.

Aspect 17: The method according to any one of aspects 10-12, wherein the effective amount of the binding agent induces lymphodepletion, and the method further comprises: administering to the subject an effective amount of a population of cells expressing a chimeric antigen receptor or a T-cell receptor (CAR/TCR).

Aspects 18: A pharmaceutical composition comprising any of the binding agents of aspects 3 or 4 and a pharmaceutically acceptable diluent.

Aspect 19: An article of manufacture comprising (a) a radiolabeled binding agent according to any of aspects 3 or 4, and (b) a label instructing the user to administer to a subject an amount of the binding agent effective to provide a therapeutic effect.

Aspect 20: The article of manufacture of aspect 19, wherein the therapeutic effect is any of: depletion of the subject's lymphocytes, ablation of the subject's myeloid cells, and inhibition or cessation of growth of CD45-expressing cells.

Aspect 21. An isolated polypeptide comprising: a conformational epitope of CD45, wherein the epitope comprises at least two amino acids in the d1 region of human CD45 as set forth in SEQ ID NO:19.

Aspect 22. The polypeptide according to aspect 21, wherein the epitope comprises at least two amino acids in the region 228-288 of human CD45 as set forth in SEQ ID NO: 20.

Aspect 23. The polypeptide according to aspect 21, wherein the epitope comprises at least two amino acids in the region 254-286 of human CD45 as set forth in SEQ ID NO: 21.

Aspect 24. The polypeptide according to aspect 21, wherein the at least two amino acids are selected from the amino acid residues V254, N257, E259, N267, N268, H285, or N286 of the sequence as set forth in SEQ ID NO: 21.

Aspect 25. The polypeptide according to aspect 21, wherein the epitope comprises the amino acid residues V254, N257, E259, N267, H285, and N286 of the sequence as set forth in SEQ ID NO: 21.

Aspect 26. Use of the epitope according to any of aspects 21 to 25 to produce a binding agent, wherein the binding agent is an antibody, antibody fragment, peptide, or small molecule capable of specifically binding the epitope.

Aspect 27. The use according to aspect 26, wherein the binding agent binds at least two of the amino acid residues V254, N257, E259, N267, N268, H285, or N286 of the sequence as set forth in SEQ ID NO: 21.

Aspect 28. An isolated binding agent comprising: an antibody, antibody fragment, peptide, or small molecule that binds to CD45 protein, wherein the binding agent binds at least two of the amino acid residues V254, N257, E259, N267, N268, H285, or N286 of the sequence as set forth in SEQ ID NO: 21, and wherein the isolated binding agent inhibits activity of the CD45 protein.

Aspect 29. The isolated binding agent according to aspect 28, wherein the isolated binding agent inhibits binding of the monoclonal antibody BC8 to the CD45 protein or is blocked from binding to the CD45 protein by the monoclonal antibody BC8.

Aspect 30. The isolated binding agent according to aspect 28, wherein the isolated binding agent comprises a radiolabel selected from the group consisting of: ³²P, ²¹¹At, ¹³¹I, ¹³⁷Cs, ⁹⁰Y, ¹⁷⁷Lu, ¹⁸⁶Re, ¹⁸⁸Re, ⁸⁹Sr, ¹⁵³Sm, ²²⁵Ac, ²¹³Bi, ²¹³Po, ²¹²Bi, ²²³Ra, ²²⁷Th, ¹⁴⁹Tb, ¹⁶⁴Cu, ²¹²Pb, ⁸⁹Zr, ⁶⁸Ga, and ¹⁰³Pd.

Aspect 31. The isolated binding agent according to aspect 28, wherein the binding agent comprises ¹³¹I or ²²⁵Ac.

Aspect 32. A method for treating a subject having a disease or disorder involving cells expressing CD45, the method comprising: administering to the subject an effective amount of an isolated binding agent comprising an antibody, antibody fragment, peptide, or small molecule that binds to CD45 protein, wherein the binding agent binds at least two of the amino acid residues V254, N257, E259, N267, N268, H285, or N286 of the sequence as set forth in SEQ ID NO: 21.

Aspect 33. The method according to aspect 32, wherein the binding agent comprises an 225Ac radiolabel, and the effective amount comprises a dose of 0.1 to 10 μCi/kg body weight of the subject.

Aspect 34. The method according to aspect 32, further comprising: transplanting allogeneic stem cells to the subject 8 to 20 days after the administration of the binding agent, wherein the effective amount of the binding agent comprises a dose sufficient to induce myeloablation.

Aspect 35. The method according to aspect 34, wherein the effective amount provides a radiation dose of greater than 8 Gy to the bone marrow of the subject.

Aspect 36. The method according to aspect 32, further comprising: administering to the subject an effective amount of a population of cells expressing a chimeric antigen receptor or T-cell receptor (CAR/TCR) 6, 7, or 8 days after the administration of the binding agent, wherein the effective amount of the binding agent comprises a dose sufficient to lymphodeplete the subject.

Aspect 37. An article of manufacture comprising: (a) a radiolabeled binding agent, and (b) a label instructing the user to administer to a subject an amount of the binding agent effective to provide a therapeutic effect, wherein the binding agent comprises an antibody, antibody fragment, peptide, or small molecule that binds to CD45 protein, wherein the binding agent binds at least two of the amino acid residues V254, N257, E259, N267, N268, H285, or N286 of the sequence as set forth in SEQ ID NO: 21, and wherein the isolated binding agent inhibits activity of the CD45 protein.

Aspect 38. The article of manufacture according to aspect 37, wherein the therapeutic effect is any of: depletion of the subject's lymphocytes, ablation of the subject's myeloid cells, and inhibition or cessation of growth of CD45-expressing cells.

Aspect 39. The article of manufacture according to aspect 37, wherein the radiolabeled binding agent comprises a radiolabel selected from the group consisting of: ³²P, ²¹¹At, ¹³¹I, ¹³⁷Cs, ⁹⁰Y, ¹⁷⁷Lu, ¹⁸⁶Re, ¹⁸⁸Re, ⁸⁹Sr, ¹⁵³Sm, ²²⁵Ac, ²¹³Bi, ²¹³Po, ²¹²Bi, ²²³Ra, ²²⁷Th, ¹⁴⁹Th, ¹⁶⁴Cu, ²¹²Pb, ⁸⁹Zr, ⁶⁸Ga, and ¹⁰³Pd.

Aspect 40. The isolated binding agent according to aspect 37, wherein the binding agent comprises ¹³¹I or ²²⁵Ac, and the amount of the binding agent effective to provide a therapeutic effect delivers a radiation dose of less than 8 Gy to the bone marrow of the subject to affect lymphodepletion or greater than 8 Gy to the bone marrow of the subject to affect myeloablation.

### EXAMPLES

### Example 1: Production of Anti-CD45 Immunoglobulin BC8

The murine anti-CD45 mAb BC8 was prepared from a hybridoma (ATCC No. HB-10507) that was initially developed by fusing mouse myeloma NS1 cells with spleen cells from a BALB/C mouse hyperimmunized with human phytohemagglutinin (PHA)-stimulated mononuclear cells. The original fused cells, after screening for microbial contaminations, were cultured using the JRH-Biosciences EXCell 300 medium supplemented with 1-2% Fetal Bovine Serum (FBS).

The hybridoma cell line was adapted for culture in a serum-free culture medium. Briefly, the cells in culture were slowly and gradually weaned of the serum albumin using the combo medium supplemented with glutamine, cholesterol, insulin and transferrin. The cells were then grown in up to 500 L scale to a density of >1 e6 cells/mL. The medium was harvested and processed for the purification of the anti-CD45 antibody using a combination of cation exchange chromatography, Protein-A chromatography, and anion exchange membrane separation. The purified antibody was concentrated by nano-filtration (30 kD cutoff). The concentration of the purified product was measured at 5.2 mg/ml and was stored at 2-8° C.

The purified antibody was characterized by SDS-PAGE, IEF and SEC-HPLC techniques. A single product peak (99.4%) was recorded with SEC-HPLC with about 0.6% aggregates. The non-reducing SDS-PAGE showed the band at 186.55 kD for the antibody. The SDS-PAGE under reduced conditions confirmed the presence of the light and the heavy chains (99.9% together).

### Example 2: Fragments of Anti-CD45 Immunoglobulin BC8

Immunoreactive agents with an epitope defined herein may be sub-fragment(s) of the antibody BC8. This includes single chain variable fraction (scFv) molecules comprising SEQ ID NO: 2 and SEQ ID NO: 3. Such molecules are typically produced in E. coli by standard production methods. Further, Fab fragments of BC8 may be alternatively produced for binding to any of the epitopes of CD45 defined herein. Exemplary Fab fragments include those defined by SEQ ID NOS: 15 and 17, which may contain SEQ ID NO: 3 and SEQ ID NO: 12. A chimeric Fab may also be generated using SEQ ID NO: 2 and SEQ ID NO: 13, and/or SEQ ID NO: 3 and SEQ ID NO: 14. These include constant regions of a human IgG molecules, such as the heavy chain of IgG1 and the light chain kappa region. Other constant regions from other IgG molecules may be used, such as from human Ig (12 and/or IgG4.

### Example 3: Sequencing of the Anti-CD45-Immunoglobulin BC8

DNA Sequence: Total RNA was isolated from the hybridoma cells following the technical manual of Trizol® Reagent. The total RNA was analyzed by agarose gel electrophoresis and was reverse transcribed into cDNA using isotype-specific anti-sense primers or universal primers following the technical manual of PrimeScript™ Ist Strand eDNA Synthesis Kit. The antibody fragments of VH, VL, CH and CL were amplified and were separately cloned into a standard cloning vector using standard molecular cloning procedures. Colony PCR screening was performed to identify clones with inserts of correct sizes. More than five single colonies with inserts of correct sizes were sequenced for each antibody fragment. The complete nucleotide sequence of the light and the heavy chains are shown in FIGS. 7 and 8.

Protein Sequencing by LC-MS/MS: The anti-CD45-antibody was sequenced using the mass spectrometry peptide mapping approach. The anti-CD45-antibody was de-glycosylated, reduced and digested with individual enzymes; trypsin, Lys-C and chymotrypsin. The peptide fragments were then analyzed by the LC-coupled mass spectrometry technique using the MS/MS fragmentation analysis approach. In the LC-MS/MS peptide mapping-based sequence, certain isomeric amino acids of the same mass may be mistaken for one another. For example, interpretation between a Leu and Ile can be difficult. The nucleotide-based sequence was used to correct for such ambiguities.

The complete predicted protein sequences of the light chain, based on the DNA sequence and standard codon usage, is shown in FIG. 7. Protein sequencing of the heavy and light chains of the BC8 antibody showed that the actual amino acid sequence differs from that predicted by the DNA sequence by only a single amino acid in the heavy chain. The complete actual sequence of the heavy chain, based on this protein sequencing, is shown in FIG. 8. The codon which codes for the amino acid at position 141 predicts an ASN-141, whereas protein sequencing of various lots of the protein purified in the lab show that the amino acid at that position is ASP-141 a certain percentage of the time (i.e., position 141 is found to be a mixture of ASP and ASN in the purified protein).

This type of post-translational modification, deamination, may depend on the cellular environment and, in some cases, has been postulated to be related to protein age (e.g., may provide a signal for protein degradation). The fact that other deaminated amino acids were not identified, however, may be indicative of an important and specific role for ASP-141. At the very least, ASP-141 may be in an exposed or accessible region on the folded protein. That is, ASN-141 may be solvent accessible and reside within a conformationally flexible region of the antibody. The effect of deamination on the biological activity of the BC8 antibody may be determined from the results of human clinical trials.

### Example 4: Optimization of MAbs/Fabs Against the Target Protein

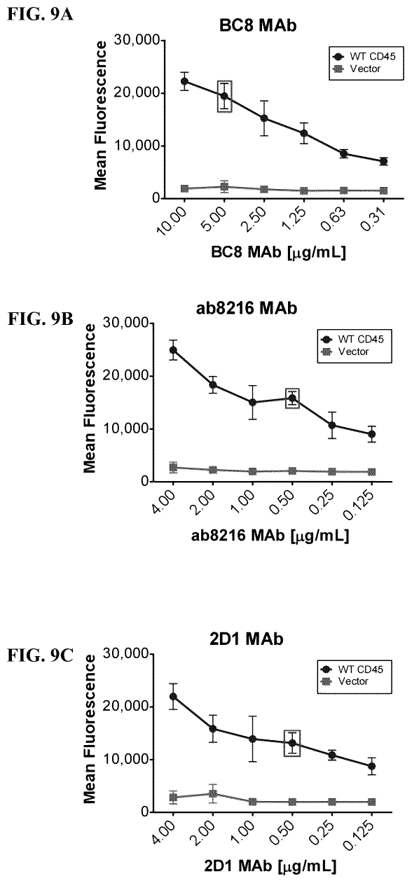

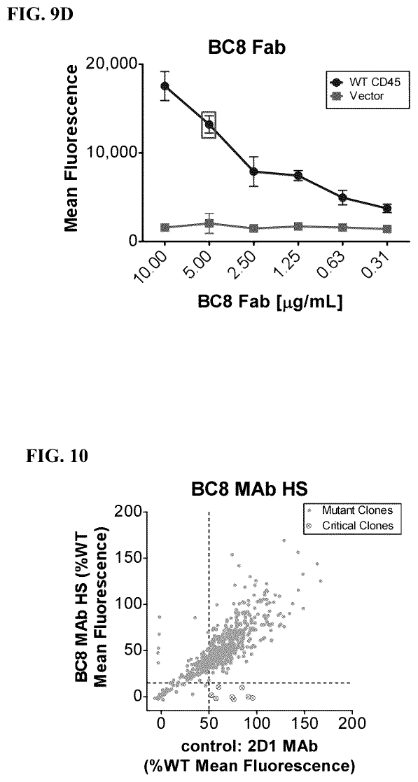

Cells were transfected with a wild-type (WT) construct of the target protein or with vector alone in 384-well format, followed by detection of cellular expression via high-throughput flow cytometry. Serial dilutions of each MAb were tested for immunoreactivity against cells expressing target protein (WT) or vector alone. With reference to FIGS. 9A-9D, the optimal screening concentration for each Mab or Fab (boxed point) was determined based on the raw signal values and signal-to-background calculations (each point represents the mean of four replicates). Optimized parameters for the screening are shown in Table 2.

### Example 5: Immunophenotypic Profiling of Anti-CD45 Antibody BC8-Comparison to Clone D12

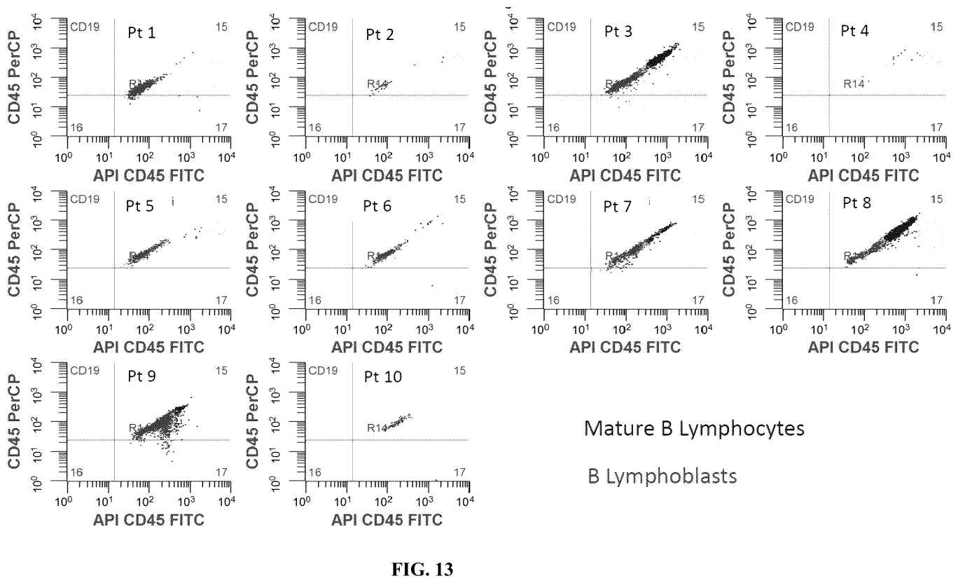

The quantitative expression of CD45 on stressed bone marrow specimens has been extensively studied using clone D12 (Becton Dickinson Biosciences, San Jose CA). The amount of this antibody on 5 reference populations of cells within the bone marrow specimens has been shown to be invariant from individual to individual, independent of age. A comparison of CD45 binding of BC8 and D12 on regenerating bone marrow cells was performed by using two different fluorophores (BC8-fluorescein, D12-peridinin chlorophyll protein) mixed together. This approach correlates intensities of these two dyes on cell populations within the bone marrow. Since cells of different lineages and maturational stages express different levels of CD45, the correlative binding of the two antibodies demonstrates the intensity relationships on diverse cell populations. By including additional antibodies in the assay it is possible to demonstrate directly the intensity relationships between the two CD45 antibodies on each major cell lineage as well as maturational difference within lineages. See for example a representative flow cytometry experiment using andti-CD19 to detect mature B lymphocytes and B lymphocytes (FIG. 13).

Normal bone marrow specimens (5 pediatric and 5 adult; Pt 1, 5, 6, 9, and 10 are pediatric; and PT 2, 3, 4, 7, and 8 are adult) were selected from clinical specimens for residual disease detection that were classified as having no evidence of disease. The cell populations included progenitor cells, mature and immature B lymphoid cells, T cells, NK cells, myeloid and erythroid progenitor cells, monocytes, maturing neutrophils, plasmacytic dendritic cells and basophils.

The intensity correlation between these two antibodies was extremely tight on all cell populations, indicating these antibodies identified the same antigen with the same intensity of reactivity. An exception was identified in these studies where the fluorescein isothiocyanate (FITC) conjugated BC8 bound to a subset of neutrophils (identified by high granularity using side scatter). This type of staining was attributed to antibody aggregates commonly produced during FITC coupling to antibodies. Without additional steps to remove such aggregates, the aggregate antibodies can bind to the Fc receptors on leukocytes, rather than through their antibody combining site.

Conclusion: the two antibodies identify the same antigen on bone marrow cells in a quantitative manner with identical reactivity on all major cell populations.

TABLE 2
Experimental parameters optimized for high-throughput flow cytometry.

| Experimental | | | | | | | | |
|---|---|---|---|---|---|---|---|---|
| Parameter | Test MAb | | Control MAb | | Control MAb | | Test Fab | |
| Cell Type | HEK-293T | | HEK-293T | | HEK-293T | | HEK-293T | |
| Fixative | None | | None | | None | | None | |
| Blocking Buffer | 10% Goat Serum | | 10% Goat Serum | | 10% Goat Serum | | 10% Goat Serum | |
| Primary Antibody | | | | | | | | |
| Name | BC8 MAb | | ab8216 MAb | | 2D1 MAb | | BC8 Fab | |
| | | | (Abcam) | | (R&D Systems) | | | |
| Target | CD45 | | CD45 | | CD45 | | CD45 | |
| Optimal Conc. | 5.00 | μg/ml | 0.50 | μg/ml | 0.50 | μg/ml | 5.0 | μg/ml |
| Incubation (25° C.) | 60 | min | 60 | min | 60 | min | 60 | min |
| Secondary Antibody | | | | | | | | |
| Target | Mouse IgG | | Mouse IgG | | Mouse IgG | | Mouse F(ab′)2 | |
| Optimal Conc. | 1:400 | | 1:400 | | 1:400 | | 1:200 | |
| | (3.75 μg/ml) | | (3.75 μg/ml) | | (3.75 μg/ml) | | (7.5 ug/ml) | |
| Incubation (25° C.) | 30 | min | 30 | min | 30 | min | 30 | min |

| Manufacturer/CAT # | Jackson lmmunoResearch/115-545-003 | | | |
|---|---|---|---|---|
| Antibody ID | AlexaFluor ® 488 AffiniPure Goat Anti-Mouse IgG (H + L) | | | |
| Wash Buffer | PBS (Ca2+, Mg2+ free) | | | |
| Signal:Background | 9:1 | 8:1 | 7:1 | 6:1 |

### Example 6: Identification of Critical Clones for MAb Binding

Binding of each test Mab/Fab to each mutant clone in the alanine scanning library was determined, in duplicate, by high-throughput flow cytometry. For each point, background fluorescence was subtracted from the raw data, which were then normalized to Mab/Fab reactivity with WT target protein. For each mutant clone, the mean binding value was plotted as a function of expression (represented by control reactivity). To identify preliminary primary critical clones (red circles), a threshold (dashed lines) of >50% WT binding to control MAb and <15% WT binding to test MAbs was applied (see FIG. 10).

### Example 7: Identification of Critical Residues for MAb Binding

Mean binding reactivities (and ranges) for all identified residues are listed in Table 3. Critical residues for MAb binding (outlined in the table) were residues whose mutations were negative for binding to test MAbs, but positive for binding to control MAbs. Although P107A met the % WT binding thresholds, it is not considered critical, as it has a relatively high range and is unable to be visualized on the crystal structure.

Residues whose mutation gave the lowest reactivities with specific antibodies are highlighted in bold and underlined in FIG. 11C. Validated critical residues represent amino acids whose side chains make the highest energetic contributions to the antibody-epitope interaction; therefore, the highlighted residues are likely the major energetic contributors to binding.

TABLE 3
Amino Acid Residues in the Conformational Epitope
Binding Reactivity (% WT)

| Mutation | BC8 MAb HS | | 2D1 MAb | |
|---|---|---|---|---|
| P107A | 9.7 | (21) | 84.5 | (26) |
| V2544 | 0.9 | (4) | 52.7 | (17) |
| N257A | −2.8 | (4) | 75.6 | (2) |
| E259A | −1.3 | (2) | 57.0 | (1) |
| N267A | −0.7 | (0) | 74.4 | (15) |
| N268A | 10.8 | (1) | 60.3 | (10) |
| H285A | −0.1 | (5) | 90.9 | (11) |
| N286A | − 1.0 | (4) | 95.4 | (10) |

### Example 8: Selection of Antibodies, Fragments, Peptides or Small Molecules that Bind the N-Terminal Epitope of the CD45 Cysteine-Rich Region

Molecules that bind to peptides comprising the epitope defined in Table 3, or any of the other epitopes define herein, and exemplified in FIGS. 12A and 12B can be selected from libraries of molecules by panning for binding to recombinant peptides containing these critical residues. For example, peptides defined by SEQ ID NO: 19, SEQ ID NO: 20, or SEQ ID NO: 21 can be readily synthesized using a peptide synthesizer. When immobilized in a tube or plate, libraries containing whole antibodies or fragments, such as single chain variable fragment (scFv) or Fab may be screened for selection of molecules that selectively bind to these peptides and thereby to the n-terminal region of the CD45 cysteine-rich region. Those that bind to the peptide will be retained after washing the non-binding phage from the tube or well. By adjusting the concentration of peptide, and rescreening the binders under increasingly stringent conditions, more specific and higher affinity binders may be obtained. Peptide libraries or small molecules may also be screened for selective binding to these peptides in a similar fashion. It is also possible to isolate antibodies that bind to the defined epitope by immunizing a suitable animal with the peptides defined in SEQ ID NO: 19, SEQ ID NO: 20, or SEQ ID NO: 21 for the generation of reactive antibodies. Suitable animals may include be mice, rats, chickens, rabbits, or llamas. In some cases, DNA encoding these peptide regions may be injected as immunogen in place of a peptide. Following the induction of an antibody response, hybridomas may be generated by the fusion of isolated antibody producing B cells from the immunized animals with an immortalized myeloma cell line using methods established in the field. Following expansion of these hybridomas, antibodies selective for binding to the immunized peptide can be selected. Methods also are known for directly isolating and cloning the genes encoding the expressed antibodies from the B cells by polymerase chain reaction following induction of an antibody response. Once cloned and produced, antibodies selective for the immunized peptide may be selected.

1-20. (canceled)

21. A method for treating a subject having a disease or disorder involving cells expressing CD45, the method comprising:
administering to the subject an effective amount of an isolated binding agent comprising a humanized antibody that binds to CD45 protein,
wherein the binding agent binds at least two amino acids in the region 254-286 of human CD45 as set forth in SEQ ID NO: 21,
wherein the binding agent is not BC8, and
wherein the binding agent inhibits binding of monoclonal antibody BC8 to the CD45 protein or is blocked from binding to the CD45 protein by the monoclonal antibody BC8.

22. The method according to claim 21, wherein the binding agent comprises ¹³¹I or ²²⁵Ac.

23. A method for treating a subject having a disease or disorder involving cells expressing CD45, the method comprising:
administering to the subject an effective amount of an isolated binding agent comprising an antibody, antibody fragment, peptide, or small molecule that binds to CD45 protein,
wherein the binding agent binds at least two amino acids in the region 254-286 of human CD45 as set forth in SEQ ID NO: 21.

24. The method according to claim 23, wherein the at least two amino acids are selected from the group consisting of amino acid residues V254, N257, E259, N267, N268, H285, or N286 of the sequence as set forth in SEQ ID NO: 21

25. The method according to claim 23, wherein the binding agent comprises an 131I or 225Ac radiolabel, and the effective amount comprises a dose of 0.1 to 10 μCi/kg body weight of the subject.

26. The method according to claim 23 comprising:
transplanting allogeneic stem cells to the subject 8 to 20 days after the administration of the binding agent,
wherein the effective amount of the binding agent comprises a dose sufficient to induce myeloablation.

27. The method according to claim 26, wherein the effective amount provides a radiation dose of greater than 8 Gy to the bone marrow of the subject.

28. The method according to claim 23 comprising:
administering to the subject an effective amount of a population of cells expressing a chimeric antigen receptor or T-cell receptor (CAR/TCR) 6, 7, or 8 days after the administration of the binding agent,
wherein the effective amount of the binding agent comprises a dose sufficient to lymphodeplete the subject.

29. The method according to claim 23, wherein the disease or disorder involving cells expressing CD45 is a malignant hematological disease or disorder.

30. The method according to claim 23, wherein the disease or disorder involving cells expressing CD45 is a non-malignant hematological disease or disorder.

31. The method according to claim 23, wherein the binding agent is not BC8.

32. The method according to claim 23, wherein the binding agent is a humanized antibody that binds to a conformational epitope of CD45 protein.

33. The method according to claim 23, wherein the binding agent comprises a radiolabel selected from the group consisting of: ³²P, ²¹¹At, ¹³¹I, ¹³⁷Cs, ⁹⁰Y, ¹⁷⁷Lu, ¹⁸⁶Re, ¹⁸⁸Re, ⁸⁹Sr, ¹⁵³Sm, ²²⁵Ac, ²¹³Bi, ²¹³Po, ²¹²Bi, ²²³Ra, ²²⁷Th, ¹⁴⁹Tb, ¹⁶⁴Cu, ²¹²Pb, ⁸⁹Zr, ⁶⁸Ga, and ¹⁰³Pd.

34. The method according to claim 23, wherein the binding agent comprises ¹³¹I.

35. The method according to claim 23, wherein the binding agent comprises ²²⁵Ac.

36. The method according to claim 23, wherein the isolated binding agent comprises an ²²⁵Ac radiolabel, and the effective amount comprises a dose of 3.7 to 370 kBq/kg (0.1 to 10 μCi/kg) body weight of the subject.

37. The method according to claim 23 comprising:
transplanting allogeneic stem cells to the subject 8 to 20 days after the administration of the binding agent,
wherein the effective amount of the binding agent comprises a dose sufficient to induce myeloablation.

38. The method according to claim 37, wherein the effective amount provides a radiation dose of greater than 8 Gy to the bone marrow of the subject.

39. The method according to claim 23, wherein the effective amount of the binding agent comprises a dose sufficient to lymphodeplete the subject.

40. A method for treating a subject having a disease or disorder involving cells expressing CD45, the method comprising:
administering to the subject an effective amount of an isolated binding agent comprising a humanized antibody that binds to CD45 protein,
wherein the binding agent binds at least two amino acids in the region 254-286 of human CD45 as set forth in SEQ ID NO: 21,
wherein the binding agent comprises ¹³¹I or ²²⁵AG, and
wherein the binding agent is not BC8.


SEQUENCE LISTING (21 sequences)

SEQ ID NO: 1 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 2 - protein, 111 aa, Mus musculus
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 3 - protein, 123 aa, Mus musculus
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSAK

SEQ ID NO: 4 - protein, 15 aa, Mus musculus
RASKSVSTSGYSYLH

SEQ ID NO: 5 - protein, 7 aa, Mus musculus
LASNLES

SEQ ID NO: 6 - protein, 9 aa, Mus musculus
QHSRELPFT

SEQ ID NO: 7 - protein, 10 aa, Mus musculus
GFDFSRYWMS

SEQ ID NO: 8 - protein, 17 aa, Mus musculus
EINPTSSTINFTPSLKD

SEQ ID NO: 9 - protein, 12 aa, Mus musculus
GNYYRYGDAMDY

SEQ ID NO: 10 - protein, 7 aa, Mus musculus
DIALTQS

SEQ ID NO: 11 - protein, 7 aa, Mus musculus
EVKLLES

SEQ ID NO: 12 - protein, 96 aa, Mus musculus
AKTTPPSVYPLAPGSAAQTDSMVTLGCLVKGYFPEPVTWTWNSGSLSSGVHTFPAVLQSDLYTLSSSVTVTSSTWPSQSITCNVAHPASSTKVDKK

SEQ ID NO: 13 - protein, 107 aa, Homo sapiens [sequence omitted]

SEQ ID NO: 14 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 15 - protein, 238 aa, Mus musculus
METDTLLLWVLLLWVPGSTGDIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - DNA, 717 bp, Mus musculus
  source 1..717: mol_type = genomic DNA
atggagacagacacactcctgttatgggtactgctgctctgggttccaggttccactggtgacattgcgctgacacagtctcctgcttccttagctgtatctctgggacagagggccaccatctcatgcagggccagcaaaagtgtcagtacatctggctatagttatctgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 17 - protein, 463 aa, Mus musculus
MDFGLIFFIVALLKGVQCEVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSAKTTPPSVYPLAPGSAAQTDSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 18 - DNA, 1392 bp, Mus musculus
  source 1..1392: mol_type = genomic DNA
atggattttgggctgattttttttattgttgctcttttaaaaggggtccagtgtgaggtgaagcttctcgagtctggaggtggcctggtgcagcctggaggatccctgaaactctcctgtgcagcctcaggattcgatttcagtagatactggatgagttgggtccggcaggctccagggaaagggctagaatggattggagagattaatccaactagcagtacgataaactttacgccatctctaaaggataaagtcttcatctccagagacaacgccaaaaatacgctgtacctgcaaatgagcaaagtgagatctgaggacacagccctttattactgtgcaagagggaactactataggtacggagatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 19 - protein, 75 aa, Homo sapiens
PTCDEKYANITVDYLYNKETKLFTAKLNVNENVECGNNTCTNNEVHNLTECKNASVSISHNSCTAPDKTLILDVP

SEQ ID NO: 20 - protein, 61 aa, Homo sapiens
CDEKYANITVDYLYNKETKLFTAKLNVNENVECGNNTCTNNEVHNLTECKNASVSISHNSC

SEQ ID NO: 21 - protein, 33 aa, Homo sapiens
VNENVECGNNTCTNNEVHNLTECKNASVSISHN